(19) United States
(12) Patent Application Publication
(10) Pub. No.: US 2022/0298455 A1
(43) Pub. Date: Sep. 22, 2022

(54) Use Of A Mannanase Enzyme In Combination With Catechol Derivatives

(71) Applicant: Henkel AG & Co. KGaA, Duesseldorf (DE)

(72) Inventors: Simone Bergmann-Dalkilic, Duesseldorf (DE); Mareile Job, Leverkusen (DE); Christian Kropf, Hilden (DE); Frank Meier, Duesseldorf (DE); Ulrich Pegelow, Duesseldorf (DE)

(21) Appl. No.: 17/493,747

(22) Filed: Oct. 4, 2021

Related U.S. Application Data
(63) Continuation of application No. PCT/EP2020/055337, filed on Feb. 28, 2020.

(30) Foreign Application Priority Data
Apr. 4, 2019 (DE) 102019204792.0

(51) Int. Cl. C11D 3/32; C12N 9/24; C11D 11/00; C11D 1/83; C11D 3/386

(52) U.S. Cl. CPC C11D 3/32; C12N 9/2491; C12Y 302/01025; C11D 11/0017; C11D 1/83; C11D 3/38636; C11D 1/22

(57) ABSTRACT

The present invention relates to aqueous surfactant compositions, in particular detergents, which contain at least one catechol compound, at least one surfactant, and at least one mannanase enzyme. The present invention further relates to the use of the surfactant composition for removing bleachable stains and to a method for washing textiles using the surfactant composition.

### FIELD OF THE INVENTION

The present invention relates to a detergent, preferably a liquid detergent, which comprises at least one catechol compound, at least one surfactant, and at least one mannanase enzyme. The present invention further relates to the use of the detergent for removing bleachable stains and a method for washing textiles using the detergent.

### BACKGROUND OF THE INVENTION

While the formulation of powdered detergents and cleaning agents containing bleaching agents no longer poses any problems, the formulation of stable liquid detergents and cleaning agents containing bleaching agents continues to be problematic. Due to the usual absence of the bleaching agent in liquid detergent and cleaning agents, stains of this kind which are normally removed, in particular due to the bleaching agents contained, are thus frequently only inadequately removed. A similar problem also exists for bleaching agent-free color detergents in which the bleaching agent is omitted in order to protect the dyes in the textiles and prevent them from fading. If there is no bleaching agent, the situation is aggravated by the fact that, instead of removing the so-called bleachable stains which are normally at least partly oxidatively removed by using peroxygen-based bleaching agent, the stain is even intensified and/or made harder to remove, which may be due to initiated chemical reactions which may consist, for example, in the polymerization of particular dyes contained in the stains.

Such problems occur in particular in stains which contain polymerizable substances. The polymerizable substances are primarily polyphenolic dyes, preferably flavonoids, in particular from the class of anthocyanidins or anthocyanins. The stains can in particular have been caused by food products or beverages which contain the corresponding dyes. The stains can in particular be stains from fruits or also vegetables or also red wine stains which in particular contain polyphenolic dyes, especially those from the class of anthocyanidins or anthocyanins.

From international patent application WO 2011/023716 A1 the use of gallic acid esters, such as propyl gallate, in detergents and cleaning agents for improved removal of stains which contain polymerizable substances, is known.

The international patent application WO 2013/092263 A1 relates to the improvement of the performance of washing and cleaning agents by using oligohydroxybenzoic acid amides.

German patent applications DE 102016214660 A1 and DE 102014222833 relate to the use of dihydroxyterephthalic acid derivatives in detergents and cleaning agents in order to improve washing or cleaning performance. The detergents and cleaning agents in these publications have an alkaline pH.

It has been shown that food products which, in addition to the polymerizable substances, also contain thickeners based on polysaccharides, such as chocolate pudding, lead to soiling that is very difficult to remove.

### BRIEF SUMMARY OF THE INVENTION

The object of the present invention was to provide a surfactant composition which removes soiling (in particular soiling caused by food products which contain polymerizable substances and polysaccharide-based thickeners) to an outstanding degree. The components should have good efficiency, and a very good washing result, in particular an excellent degree of whiteness, should be achieved.

In this regard, the inventors of the present invention have found that the combination of special catechol compounds with mannanase leads to improved stain removal, especially when the soiling to be removed was caused by food products which contain polymerizable substances and polysaccharide-based thickeners.

The present invention therefore firstly relates to a surfactant composition, especially as a laundry detergent, which:
comprises at least one catechol compound of the formula (I) wherein

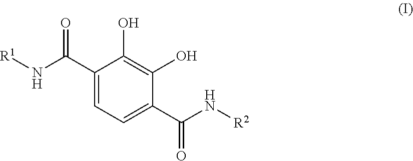

R¹ and R² represent, independently of one another, a hydrocarbon radical having 1 to 20 carbon atoms that is optionally substituted by at least one radical selected from hydroxy, (C₁-C₄)-alkoxy, (C₁-C₄)-alkoxy(CH₂CH₂O)ₙ—, —NR′R″ or —N+R′R″R′″X′, wherein n=1 to 10, R′, R″ and R′″ represent, independently of one another, H or a linear or branched aliphatic hydrocarbon radical having 1 to 3, preferably 1 to 2, carbon atoms and X⁻ represents an anion;
   at least one surfactant, preferably based on the total weight of the surfactant composition in a total amount of 2 to 70 wt.-%, in particular of 10 to 65 wt. %, particularly preferably from 15 to 60 wt. %;
   at least one mannanase enzyme;
   optionally other active ingredients; and
   water based on the total weight of the surfactant composition from 1 to 50 wt. %, preferably 2 to 30 wt. %, particularly preferably 3 to 25 wt. %, very particularly preferably 5 to 25 wt. %, in particular 5 to 35 wt. %, preferably 5 to 30 wt. %, particularly preferably 5 to 25 wt. %, very particularly preferably 8 to 15 wt. %, based on the total weight of the surfactant composition.

The water content as defined herein refers to the water content as determined by means of Karl Fischer titration (Angewandte Chemie 1935, 48, 394-396; ISBN 3-540-12846-8 Eugen Scholz).

“Liquid,” when used herein in relation to the compositions according to the invention, includes all compositions which are flowable under standard conditions (20° C., 1013 mbar) and in particular also includes gels and pasty compositions. In particular, the term also includes non-Newtonian liquids which have a yield point. Granular mixtures (flowable solids such as powder or granulate mixtures) are known not to be liquids and therefore not included.

Unless otherwise indicated, all stated amounts indicated in connection with the constituents of the detergent described herein refer to wt. %, in each case based on the total weight of the composition. Moreover, stated amounts of this kind that refer to at least one constituent always refer to the total amount of this type of constituent contained in the detergent, unless explicitly indicated otherwise. This means that stated amounts of this kind, for example in connection with “at least one non-ionic surfactant,” refer to the total amount of non-ionic surfactant contained in the detergent.

“At least one,” as used herein, refers to 1 or more, for example 1, 2, 3, 4, 5, 6, 7, 8, 9 or more. In connection with constituents of the compositions described herein, this information does not refer to the absolute amount of molecules, but rather to the type of the constituent. “At least one non-ionic surfactant” therefore signifies, for example, one or more different non-ionic surfactants, i.e. one or more different types of non-ionic surfactants. Together with stated amounts, the stated amounts refer to the total amount of the correspondingly designated type of constituent, as defined above.

“Substantially free,” as used herein, means that the particular compound (e.g. transition metal ion), relative to the weight of the component (e.g. coloring dye) or composition (e.g. detergent) contained in this compound, is contained in the relevant component or composition in less than 0.01 wt. %, preferably 0.001 wt. %, more preferably 0.0001 wt. %, and most preferably not at all.

If compounds are described as “substituted” in the present invention, the possible substituents are known to a person skilled in the art. Particularly preferably, unless explicitly stated otherwise, the substituents are selected from —F, —Cl, —Br, —I, —OH, ═O, —OR¹, —NH₂, —NHR¹, —NR¹₂ and —COOR¹, wherein R¹ is an alkyl radical having 1 to 10 carbon atoms.

According to the present invention, several compositions, then referred to as components, can be spatially separated from one another.

In the present invention, the terms detergent and composition are used synonymously.

If the invention relates to a multi-component detergent, the further components can be conventional cleaning agents or a composition according to the present invention; the further components can also be solid or in the form of granules. It is preferred that all components are liquid.

If further liquid components are present, they can be defined as K1 or K2 are.

Unless explicitly stated otherwise, if molecular masses or relative molecular masses or molar masses are described in the present invention, the number average molecular weight M_{N}, which can be determined by means of gel permeation chromatography using polystyrene standards, is used.

Compounds of the general formula (I) preferably have a solubility in fully demineralized water of pH 4 at 20° C. of at least 10 mg/l, in particular at least 20 mg/l.

It is preferred here if, based on the total weight of the composition, the at least one catechol compound according to formula (I) is contained in a total amount of 0.1 wt. % to 90 wt. %, in particular 0.5 wt. % to 50 wt. %, preferably from 1.0 wt. % to 40 wt. %, more preferably from 2.5 wt. % to 20 wt. %.

The hydrocarbon radicals having 1 to 20 carbon atoms in formula (I) can be linear or branched, saturated or unsaturated, cyclic or alicyclic or aromatic.

Preferred compositions are those which contain at least one catechol compound of the formula (I) in which the radicals R¹ and R² independently of one another represent an alkyl group (such as methyl, ethyl, n-propyl or i-propyl), an alkoxyalkyl group (such as methoxyethyl, methoxypropyl, (2-methoxy) ethoxyethyl, ethoxyethyl, ethoxypropyl or (2-ethoxy) ethoxyethyl), a hydroxyalkyl group (such as 2-hydroxyethyl, 3-hydroxypropyl, 2-hydroxypropyl, 1,2-dihydroxypropyl), a hydroxyalkyloxyalkyl group (such as 2-hydroxyethoxyethyl), (N-hydroxyethyl)-aminoethyl, (N-methoxyethyl)-aminoethyl or (N-ethoxyethyl)-aminoethyl, or an aromatic group (such as phenyl or benzyl).

Preferred alkyl groups in formula (I) are linear (C₁-C₁₀)-alkyl groups or branched (C₃-C₁₀)-alkyl groups, C₅-C₆ cycloalkyl.

It is preferred according to the invention if the radicals R¹ and R² in formula (I) are identical.

Further preferred selected compounds from the group of the catechols is at least one compound of the general formula (I-a),

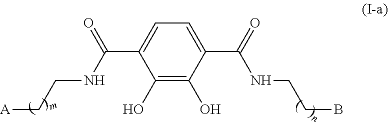

where m and n represent, independently of one another, 0 to 5 and A and B represent, independently of one another, a hydrogen atom, —NR¹R² and —N⁺R¹R²R³X⁻, and R¹, R² and R³ represent, independently of one another, H or a linear or branched aliphatic hydrocarbon radical having 1 to 3, preferably 1 to 2, carbon atoms, and X⁻ represents an anion.

Preferred compounds of general formula (l-a) are those in which A and B are identical.

X⁻ is preferably selected from the group comprising lactate, citrate, tartrate, succinate, perchlorate, tetrafluoroborate, hexafluorophosphate, alkyl sulfonate, alkyl sulfate, hydrogen sulfate, sulfate, dihydrogen phosphate, hydrogen phosphate, phosphate, isocyanate, rhodanide, nitrate, fluoride, chloride, bromide, hydrogen carbonate and carbonate, and mixtures of at least two of these, wherein it is possible to ensure the charge balance in the presence of polyvalent anions by the presence of a corresponding plurality of cationic basic structures of general formula (I) or optionally by the presence of additional cations such as sodium or ammonium ions. According to the invention, it is preferred if, according to formula (l-a), A and B represent a hydrogen atom.

The most preferred catechol compounds of formula (Formula (I) are the compounds of formulas (l-b) and/or (l-c).

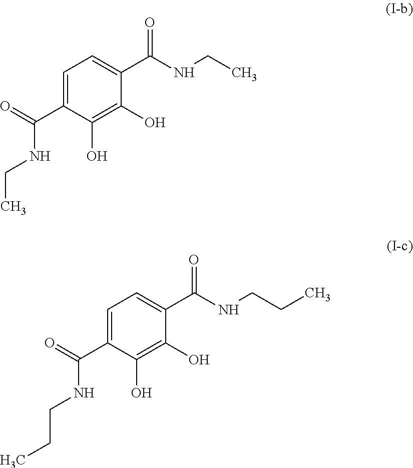

The composition according to the invention also necessarily contains at least one surfactant. It is again particularly preferred if surfactant is contained in a total amount of from 10 to 70 wt. %, in particular 20 to 65 wt. %, very particularly preferably 30 to 65 wt. %, most preferably 30 to 60 wt. %, based on the weight of the surfactant composition according to the invention.

The group of surfactants includes the non-ionic, anionic, cationic and amphoteric surfactants. According to the invention, the composition can comprise one or more of the surfactants mentioned. The composition particularly preferably comprises at least one or more anion surfactants (anionic surfactants) which are particularly preferably contained in a total amount of from 15 to 50 wt. %, in particular from 20 to 40 wt. %, based on the weight of the composition.

The at least one anionic surfactant is preferably selected from the group comprising C₉-C₁₃ alkylbenzene sulfonates, olefin sulfonates, C₁₂-C₁₈ alkane sulfonates, ester sulfonates, alk(en)yl sulfates, fatty alcohol ether sulfates and mixtures thereof. It has been found that these sulfonate and sulfate surfactants are particularly well suited to preparing stable liquid compositions. Surfactant compositions which comprise C₉-C₁₃-alkylbenzene sulfonates and fatty alcohol ether sulfates as the anionic surfactant have particularly good dispersing properties. Surfactants of the sulfonate type that can be used are preferably C₉-C₁₃-alkylbenzene sulfonates, olefin sulfonates, i.e. mixtures of alkene and hydroxyalkane sulfonates, and disulfonates, as obtained, for example, from C₁₂-C₁₈ monoolefins having a terminal or internal double bond by way of sulfonation with gaseous sulfur trioxide and subsequent alkaline or acid hydrolysis of the sulfonation products. C₁₂-C₁₈ alkane sulfonates and the esters of a-sulfofatty acids (ester sulfonates) are also suitable, for example the α-sulfonated methyl esters of hydrogenated coconut, palm kernel or tallow fatty acids.

The alkaline salts and in particular the sodium salts of the sulfuric acid half-esters of C₁₂-C₁₈ fatty alcohols, for example from coconut fatty alcohol, tallow fatty alcohol, lauryl alcohol, myristyl alcohol, cetyl alcohol or stearyl alcohol, or of C₁₀-C₂₀ oxo alcohols and the half-esters of secondary alcohols having these chain lengths, are preferred as alk(en)yl sulfates. From a washing perspective, C₁₂-C₁₆ alkyl sulfates, C₁₂-C₁₅ alkyl sulfates and C₁₄-C₁₅ alkyl sulfates are preferred. 2,3-alkyl sulfates are also suitable anionic surfactants.

The salts of the sulfuric acid half-esters of fatty alcohols having 12 to 18 C atoms, for example from coconut fatty alcohol, tallow fatty alcohol, lauryl alcohol, myristyl alcohol, cetyl alcohol or stearyl alcohol, or of the oxo alcohols having 10 to 20 C atoms and the half-esters of secondary alcohols having these chain lengths, are preferred as alk(en)yl sulfates. From a washing perspective, the alkyl sulfates having 12 to 16 C atoms, alkyl sulfates having 12 to 15 C atoms and alkyl sulfates having 14 and 15 C atoms are preferred. 2,3-alkyl sulfates are also suitable anionic surfactants.

Fatty alcohol ether sulfates, such as the sulfuric acid monoesters of straight-chain or branched C7-C2i alcohols ethoxylated with 1 to 6 mol ethylene oxide, such as 2-methyl-branched C9-11 alcohols having, on average, 3.5 mol ethylene oxide (EO) or C12-18 fatty alcohols having 1 to 4 EO, are also suitable. Alkyl ether sulfates of formula (A-1) are preferred.

R¹—O—(AO)ₙ—S0₃—X⁺ (A-1)

In this formula (A-1), R¹ represents a linear or branched, substituted or unsubstituted alkyl radical, preferably a linear, unsubstituted alkyl radical, particularly preferably a fatty alcohol radical. Preferred radicals R¹ of formula (A-1) are selected from decyl, undecyl, dodecyl, tridecyl, tetradecyl, pentadecyl, hexadecyl, heptadecyl, octadecyl, nonadecyl, eicosyl radicals and mixtures thereof, the representatives having an even number of C atoms being preferred. Particularly preferred radicals R¹ of formula (A-1) are derived from fatty alcohols having 12 to 18 C atoms, for example from coconut fatty alcohol, tallow fatty alcohol, lauryl alcohol, myristyl alcohol, cetyl alcohol or stearyl alcohol, or from oxo alcohols having 10 to 20 C atoms.

In formula (A-1), AO represents an ethylene oxide (EO) or propylene oxide (PO) group, preferably an ethylene oxide group. The index n in formula (A-1) is an integer of from 1 to 50, preferably from 1 to 20, and in particular from 2 to 10. Very particularly preferably, n is 2, 3, 4, 5, 6, 7 or 8. X is a monovalent cation or the n-th part of an n-valent cation, the alkali metal ions, including Na⁺ or K⁺, being preferred in this case, wherein Na⁺ is most preferred. Other cations X⁺ may be selected from NH₄⁺, ½Zn²⁺, ½Mg²⁺, ½Ca²⁺, ½Mn²⁺, and mixtures thereof.

Particularly preferred laundry detergents contain an alkyl ether sulfate selected from fatty alcohol ether sulfates of formula A-2

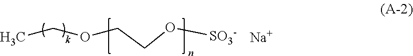

where k=11 to 19, and n=2, 3, 4, 5, 6, 7 or 8. Very particularly preferred representatives are Na fatty alcohol ether sulfates having 12 to 18 C atoms and 2 EO (k=11 to 13, n=2 in formula A-1). The degree of ethoxylation indicated represents a statistical average that can correspond to an integer or a fractional number for a specific product. The degrees of alkoxylation specified represent statistical averages that can correspond to an integer or a fractional number for a specific product. Preferred alkoxylates/ethoxylates have a narrowed homolog distribution (narrow range ethoxylates, NRE).

It is preferred that the composition contain a mixture of sulfonate and sulfate surfactants. In a particularly preferred embodiment, the composition contains C9-13 alkylbenzene sulfonates and optionally also fatty alcohol ether sulfates as the anionic surfactant.

It is very particularly preferred for the composition to contain at least one anionic surfactant of formula (A-3),

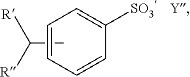

where
R′ and R″ signify, independently of one another, H or alkyl, and together contain 9 to 19, preferably 9 to 15 and in particular 9 to 13, C atoms, and Y⁺ is a monovalent cation or the nth part of an n-valent cation (in particular Na⁺).

In addition to the anionic surfactant, the composition can also contain soaps. Saturated and unsaturated fatty acid soaps are suitable, such as the salts of lauric acid, myristic acid, palmitic acid, stearic acid, (hydrogenated) erucic acid and behenic acid, and in particular soap mixtures derived from natural fatty acids, such as coconut, palm kernel, olive oil or tallow fatty acids.

The anionic surfactants, and the soaps, can be present in the form of the sodium, potassium, magnesium or ammonium salts thereof. The anionic surfactants are preferably present in the form of the ammonium salts thereof, wherein the ammonium ion is derived from at least one (C₂-C₆)-alkanolamine. Further preferred counterions for the anionic surfactants are also the protonated forms of choline, triethylamine, monoethanolamine, triethanolamine or methylethylamine.

The composition can (preferably together with at least one anionic surfactant) also have at least one nonionic surfactant. The non-ionic surfactant comprises alkoxylated fatty alcohols, alkoxylated fatty acid alkyl esters, fatty acid amides, alkoxylated fatty acid amides, polyhydroxy fatty acid amides, alkylphenol polyglycol ethers, amine oxides, alkyl polyglucosides, and mixtures thereof. It is again particularly preferred if non-ionic surfactant is contained in a total amount of from 10 to 40 wt. %, in particular 15 to 35 wt. %, based on the weight of the composition according to the invention.

Alkoxylated, advantageously ethoxylated, in particular primary alcohols having preferably 8 to 18 C atoms and, on average, 4 to 12 mol ethylene oxide (EO) per mol of alcohol, in which the alcohol radical can be linear or preferably methyl-branched in the 2 position, or can contain linear and methyl-branched radicals in the mixture, as are usually present in oxo alcohol radicals, are preferably used as the non-ionic surfactant. However, alcohol ethoxylates having linear radicals of alcohols of native origin having 12 to 18 C atoms, for example of coconut, palm, tallow fatty or oleyl alcohol, and, on average, 5 to 8 EO per mol of alcohol are particularly preferred. Preferred ethoxylated alcohols include, for example, C12-14 alcohols having 4 EO or 7 EO, C9-11 alcohols having 7 EO, C13-15 alcohols having 5 EO, 7 EO or 8 EO, C₁₂-C₁₈ alcohols having 5 EO or 7 EO, and mixtures thereof. The degrees of ethoxylation indicated represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used.

Examples of these are tallow fatty alcohols having 14 EO, 25 EO, 30 EO, or 40 EO. Non-ionic surfactants that contain EO and PO (propylene oxide) groups together in the molecule can also be used according to the invention. Furthermore, a mixture of a (more highly) branched ethoxylated fatty alcohol and an unbranched ethoxylated fatty alcohol, such as a mixture of a C₁₆-C₁₈ fatty alcohol having 7 EO and 2-propylheptanol having 7 EO, is also suitable. The detergent particularly preferably contains a C₁₂-C₁₈ fatty alcohol having 7 EO or a C₁₃-C₁₅ oxo alcohol having 7 EO as the non-ionic surfactant.

The composition particularly preferably contains at least one nonionic surfactant according to formula (N-1)

R³—O—(XO)ₘ—H, (N-1)

where
R³ represents a linear or branched C₈-C₁₈ alkyl group, an aryl radical or an alkyl aryl radical, XO, independently from one another, represents ethylene oxide (EO) or propylene oxide (PO) group, and
m represents an integer from 1 to 50.

In the above formula (N-1), R¹ represents a linear or branched, substituted or unsubstituted alkyl radical. In a preferred embodiment of the present invention, R¹ is a linear or branched alkyl radical having 5 to 30 C atoms, preferably 7 to 25 C atoms, and in particular 10 to 19 C atoms. Preferred radicals R¹ are selected from decyl, undecyl, dodecyl, tridecyl, tetradecyl, pentadecyl, hexadecyl, heptadecyl, octadecyl, nonadecyl radicals and mixtures thereof, wherein the representatives having an even number of C atoms are preferred. Particularly preferred radicals R¹ are derived from fatty alcohols having 12 to 19 C atoms, for example from coconut fatty alcohol, tallow fatty alcohol, lauryl alcohol, myristyl alcohol, cetyl alcohol or stearyl alcohol, or from oxo alcohols having 10 to 19 C atoms.

AO of formula (N-1) is an ethylene oxide (EO) group or propylene oxide (PO) group, preferably an ethylene oxide group. The index m in formula (N-1) is an integer of from 1 to 50, preferably 2 to 20, and more preferably 2 to 10. In particular, m is 3, 4, 5, 6 or 7. The composition according to the invention may contain mixtures of non-ionic surfactants which have different degrees of ethoxylation. Surfactants having degrees of alkoxylation/ethoxylation of at least 5 are preferred.

In summary, particularly preferred fatty alcohol alkoxylates are those of formula

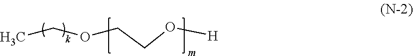

where k=9 to 17, and m=3, 4, 5, 6, or 7. Very particularly preferred representatives are fatty alcohols having 10 to 18 C atoms and having 7 EO (k=11-17, m=7 in formula N-2).

Fatty alcohol- or oxo alcohol ethoxylates of this kind are available under the trade names Dehydol® LT7 (BASF), Lutensol® AO7 (BASF), Lutensol® M7 (BASF), and Neodol® 45-7 (Shell Chemicals).

The composition according to the invention also necessarily contains at least one mannanase enzyme.

The following terms and definitions also apply in the context of the present application with a view to the definition of enzymes.

At the protein level, “variant” is the term corresponding to “mutant” at the nucleic acid level. The precursor or starting molecules can be wild-type enzymes, i.e. those which are obtainable from natural sources. They can also be enzymes which are already variants in themselves, i.e. which have already been modified compared to the wild-type molecules. These include, for example, point mutants, those having changes in the amino acid sequence, over a plurality of positions or longer contiguous regions, or also hybrid molecules which are composed of mutually complementary portions of different wild-type enzymes.

Amino acid exchanges are understood to mean substitutions of one amino acid for another amino acid. According to the invention, substitutions of this kind are indicated by the name of the positions at which the exchange takes place, optionally combined with the relevant amino acids, in the internationally used one-letter codes. “Exchange at position 320” means, for example, that a variant in the position which, in the sequence of the reference protein, comprises the position 320, comprises a different amino acid. Exchanges of this kind are usually carried out at the DNA level via mutations of individual base pairs (see above). “R320K” means, for example, that the reference enzyme at position 320 has the amino acid arginine, while the variant under consideration has the amino acid lysine at the position that can be homologated with it. “320K” means that any, e.g. usually a naturally predetermined, amino acid at a position which corresponds to position 320 is replaced by a lysine which is located precisely at this point in the present molecule. “R320K, L” means that the amino acid arginine is replaced by lysine or leucine at position 320. “R320X” means that the amino acid arginine is replaced in principle by any other amino acid at position 320.

In principle, the amino acid exchanges according to the invention and designated by the present application are not restricted to the fact that they are the only exchanges in which the variant in question differs from the wild-type molecule. It is known from the prior art that the advantageous properties of individual point mutations can complement one another. Thus, embodiments of the present invention include all variants which, in addition to other exchanges with respect to the wild-type molecule, also have the exchanges according to the invention.

Furthermore, in principle it does not matter in what order the particular amino acid exchanges have been carried out, i.e. whether a corresponding point mutant is further developed according to the invention or whether a variant according to the invention is first generated from a wild-type molecule which is developed further in accordance with other teachings to be found in the prior art. A plurality of exchanges can also be carried out simultaneously in a mutagenesis approach, for example according to the invention and other teaching together.

The identity of nucleic acid or amino acid sequences is determined by a sequence comparison. Such a comparison is made in that similar sequences in the nucleotide sequences or amino acid sequences are assigned to one another. This sequence comparison is preferably carried out on the basis of the BLAST algorithm established in the prior art and commonly used (cf. for example Altschul, S. F., Gish, W., Miller, W., Myers, E W & Lipman, D. J. (1990) “Basic local alignment search tool.” J. Mol. Biol. 215: 403-410, and Altschul, Stephan F., Thomas L. Madden, Alejandro A. Schaffer, Jinghui Zhang, Hheng Zhang, Webb Miller, and David J. Lipman (1997): “′Gapped BLAST and PSI-BLAST: a new generation of protein database search programs”; Nucleic Acids Res., 25, pp. 3389-3402) and occurs in principle by similar sequences of nucleotides or amino acids in the nucleic acid or amino acid sequences being assigned to one another. The assignment of the relevant positions shown in a table is referred to as an alignment. Another algorithm available in the prior art is the FASTA algorithm. Sequence comparisons (alignments), in particular multiple sequence comparisons, are created using computer programs. The Clustal series (cf. for example Chenna et al. (2003): Multiple sequence alignment with the Clustal series of programs. Nucleic Acid Research 31, 3497-3500), T-Coffee (cf. for example Notredame et al. (2000): T-Coffee: A novel method for multiple sequence alignments. J. Mol. Biol. 302, 205-217), or programs based on these programs or algorithms, are frequently used. Clustal (cf. for example Chenna et al. (2003): Multiple sequence alignment with the Clustal series of programs. Nucleic Acid Research 31, 3497-3500), T-Coffee (cf. for example Notredame et al. (2000): T-Coffee: A novel method for multiple sequence alignments. J. Mol. Biol. 302, 205-217) as well as BLAST or FASTA for database searches, or programs based on these programs or algorithms, are frequently used. In the context of the present invention, sequence comparisons and alignments are preferably created with the computer program Vector NTI® Suite 10.3 (Invitrogen Corporation, 1600 Faraday Avenue, Carlsbad, Calif., USA) with the predefined default parameters.

Such a comparison allows conclusions to be drawn regarding the similarity of the compared sequences. It is usually given in percent identity, i.e. the proportion of identical nucleotides or amino acid remnants in said sequences or in an alignment of corresponding positions. The broader concept of homology takes conserved amino acid exchanges into account in the case of amino acid sequences, i.e. amino acids having similar chemical activity, since they usually perform similar chemical activities within the protein. Therefore, the similarity between the compared sequences can also be expressed in percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small regions. Unless stated otherwise, however, identity or homology information in the present application relates to the entire length of the particular nucleic acid or amino acid sequence indicated.

Fragments are understood to mean all polypeptides, proteins or peptides which are smaller than corresponding comparison proteins or those which correspond to completely translated genes and can, for example, also be obtained synthetically. On the basis of their amino acid sequences, they can be assigned to the relevant complete comparison proteins. For example, they can assume the same spatial structures or exercise proteolytic activities or partial activities, such as complexing a substrate. Fragments and deletion variants of starting proteins are in principle similar; while fragments tend to represent smaller fragments, the deletion mutants tend to lack only short areas (possibly only one or more amino acids). For instance, it is possible to delete individual amino acids at the termini or in the loops of the enzyme without the enzymatic activity being lost or diminished in the process.

All enzyme quantities refer to active protein. The protein concentration can be determined using known methods, for example the BCA method (bicinchoninic acid; 2,2′-bichinolyl-4,4′-dicarboxylic acid) or the Biuret method (Gornall at al., J. Biol. Chem. 177 (1948): 751-766). The active protein concentration can be determined in this regard by titrating the active centers using a suitable irreversible inhibitor and determining the residual activity (cf. M. Bender et al., J. Am. Chem. Soc. 88, 24 (1966): 5890-5913).

### DETAILED DESCRIPTION OF THE INVENTION

In general, the enzymes contained in a composition according to the invention can be adsorbed on carrier substances and/or embedded in coating substances to protect the enzymes from premature inactivation. In the compositions described herein, the enzymes to be used may furthermore be formulated together with accompanying substances, for example from fermentation. In liquid formulations, the enzymes are preferably used as enzyme liquid formulations.

The enzymes are generally not provided in the form of pure protein, but rather in the form of stabilized, storable and transportable preparations. These pre-packaged preparations include, for example, the solid preparations obtained through granulation, extrusion, or lyophilization or, in particular in the case of liquid or gel agents, solutions of the enzymes, which are advantageously maximally concentrated, have a low water content, and/or are supplemented with stabilizers or other auxiliaries.

Compositions according to the invention can be added to the obtained enzymes in any form established according to the prior art. These include in particular the solid preparations which are obtained by granulation, extrusion or lyophilization and are advantageously as concentrated as possible, low in water and/or mixed with stabilizers. In an alternative dosage form, the enzymes can also be encapsulated, for example by spray-drying or extruding the enzyme solution together with a preferably natural polymer or in the form of capsules, for example those in which the enzymes are enclosed in a set gel, or in those of the core-shell type in which an enzyme-containing core is coated with a water-, air-, and/or chemical-impermeable protective layer. Further active ingredients such as stabilizers, emulsifiers, pigments, or dyes can additionally be applied in overlaid layers. Such capsules are applied using methods which are known per se, for example by shaking or rolling granulation or in fluidized bed processes. Such granules are advantageously low in dust, for example due to the application of polymeric film-formers, and stable in storage due to the coating.

Moreover, it is possible to formulate two or more enzymes together, such that a single granule exhibits a plurality of enzyme activities.

In various embodiments, the agent according to the invention can have one or more enzyme stabilizers. Therefore, the agent according to the invention may further contain an enzyme stabilizer, for example selected from the group consisting of sodium formate, sodium sulfate, lower aliphatic alcohols and boric acid, as well as esters and salts thereof. Of course, two or more of these compounds can also be used in combination. The salts of the compounds mentioned can also be used in the form of hydrates, such as, for example, sodium sulfate decahydrate.

A mannanase enzyme contained in the composition according to the invention catalyzes the hydrolysis of 1,4-beta-D-mannosidic bonds in mannans, galactomannans, glucomannans and galactoglucomannans as part of its mannanase activity. Said mannanase enzymes according to the invention are classified according to the enzyme nomenclature as EC 3.2.1.78. Synonyms for these mannanase enzymes according to the invention are galactomannanase, mannan-endo-1,4β-mannosidase, β-mannanase or endo-1,4-mannanase.

“Mannans” are polysaccharides containing mannose, which are composed of a mannose basic structure, in which the mannose units are linked via β-1,4-glycosidic bonds, with side chains of galactose units, which are linked via α-1,6-glycosidic links are attached to the basic structure. “Glucomannans” are polysaccharides with a backbone of more or less regularly changing β-1,4-glycosidically linked mannose and glucose units. “Galactomannans” and “Galactoglucomannans” are mannans and glucomannans with α-1,6-glycosidically linked galactose side chains.

As used herein, an enzyme has “mannanase activity” if it has mannan-degrading properties. “Degradation” or “modification” as used herein means that mannose units from the mannan polysaccharide are hydrolyzed by mannanase. The mannan-degrading activity of the polypeptides according to the present invention can be determined according to the standard test methods known in the art. Example 7 of WO 2018/184767 A1 shows an example of a standard method for determining the mannanase activity. In a further test method, a test solution is placed into holes in an agar plate that have a diameter of 4 mm and contain 0.2 wt. % of AZGL galactomannan (carob), i.e. substrate for the endo-1,4-beta-D-mannanase essay, available under catalog number I-AZGMA from Megazyme (http://www.megazyme.com), is provided.

The mannanase activity of a polypeptide or enzyme can be determined according to testing methods known from literature. For example, a test solution is placed into holes in an agar plate that have a diameter of 4 mm and contain 0.2 wt. % of AZGL galactomannan (carob), i.e. substrate for the endo-1,4-beta-D-mannanase essay, available under catalog number I-AZGMA from Megazyme (http://www.megazyme.com), is provided.

The first mannanases that are stable in detergents and cleaning agents have recently been found. Because mannanases break the β-1,4-glycosidic bond(s) between mannose units and break down the mannan into smaller, more water-soluble carbohydrate fragments, they help to remove stains containing mannans and also reduce the risk of de-accumulation.

Suitable mannanases are, for example Bacillus subtilis endo-β-mannanase (see e.g. U.S. Pat. No. 6,060,299 and WO 99/64573), Bacillus sp. 1633 endo-β-mannanase (see e.g. U.S. Pat. No. 6,566,114 and WO 99/64619), Bacillus sp. AAI12 endo-β-mannanase (see e.g. U.S. Pat. No. 6,566,114 and WO 99/64619), Bacillus sp. AA349 endo-β-mannanase (see e.g. U.S. Pat. No. 6,566,114 and WO 99/64619), Bacillus agaradhaerens NCIMB 40482 endo-β-mannanase (see e.g. U.S. Pat. No. 6,566,114 and WO 99/64619), Bacillus halodurans endo-β-mannanase, Bacillus clausii endo-β-mannanase (see e.g. U.S. Pat. No. 6,566,114 and WO 99/64619), Bacillus licheniformis endo-β-mannanase (see e.g. U.S. Pat. No. 6,566,114 and WO 99/64619), Humicola insolens endo-β-mannanase (see e.g. U.S. Pat. No. 6,566,114 and WO 99/64619) and Caldocellulosiruptor sp. endo-β-mannanase (see e.g. U.S. Pat. No. 6,566,114 and WO 99/64619), and Paenibacillus sp. (WO 2017/079751 A1).

The compositions according to the invention preferably contain, based on the total weight of the composition, mannanase enzyme in a total amount of from 0.01 to 2.5 wt. %, in particular from 0.02 to 1.0 wt. %, very particularly preferably from 0.02 to 0.7 wt. %.

Mannanase polypeptides from strains of the Thermoanaerobacter group, such as Caldicellulosiruptor, are preferably suitable according to the invention. Mannanase polypeptides of the fungi Humicola or Scytalidium, in particular of the species Humicola insolens or Scytalidium thermophilum, can also be used in the context of the invention.

It is particularly preferred according to the invention if the detergents according to the invention, as a mannanase enzyme, contains at least one mannanase polypeptide from gram-positive alkalophilic strains of Bacillus, in particular selected from at least one representative of the group of Bacillus subtilis, Bacillus lentus, Bacillus clausii, Bacillus agaradhaerens, Bacillus brevis, Bacillus stearothermophilus, Bacillus alkalophilus, Bacillus amyloliquefaciens, Bacillus coagulans, Bacillus circulans, Bacillus lautus, Bacillus thuringiensis, Bacillus cheniformis, and Bacillus sp., particularly preferably selected from at least one representative of the group of Bacillus sp. 1633, Bacillus sp. AAI12, Bacillus clausii, Bacillus agaradhaerens and Bacillus licheniformis.

A preferred mannanase enzyme according to the invention is selected from at least one representative from the group that is formed from
i) polypeptides which comprise an amino acid sequence of which the sequence is at least 90% (in order of increasing preference at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7% or 99.8%) identical to the polypeptide according to SEQ ID no.1 (cf. sequence protocol), and
ii) Polypeptides which are a fragment of (i).

It is in turn preferred if said preferred mannanase is contained in the composition according to the invention in a total amount of from 0.01 to 5.0 wt. %, in particular 0.2 to 2.0 wt. %, in each case based on the total weight of the composition.

In the case of the mannanase preferably to be used and analogously to the definition mentioned above, the fragment defined above under (ii) is understood to mean all polypeptides, proteins or peptides which are smaller than the polypeptides falling under (i) or those which correspond to fully translated genes, and can also be obtained synthetically, for example. On the basis of their amino acid sequences, they can be assigned to the relevant complete proteins. For example, they can assume the same structures or exert proteolytic activities or partial activities, such as, for example, complexing a substrate. Fragments and deletion variants of starting proteins are in principle similar; while fragments tend to represent smaller fragments, the deletion mutants tend to lack only short regions (possibly only one or more amino acids). For instance, it is possible to delete individual amino acids at the termini or in the loops of the enzyme without the enzymatic activity being lost or diminished in the process. Particularly preferred are deletions of in particular 1, up to 2, up to 3, up to 4, up to 5, in particular up to 10, preferably up to 20, particularly preferably up to 30 amino acids on the N-terminal side of the polypeptide (preferred of the polypeptide of SEQ. ID no.1). The enzymatic activity of the mannanase is preferably not reduced or reduced only to a small extent, in particular only up to a reduction of 15% of the activity of the original enzyme (particularly preferred for mannanase according to SEQ. 1D no. 1).

Particularly preferably, the mannanase is selected from at least one representative of the group formed from polypeptides which comprise an amino acid sequence of which the sequence is at least 90% (in order of increasing preference at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7% or 99.8%) identical to the polypeptide of the positions 31 to 490 according to SEQ ID no.1 (see sequence protocol).

Very particularly preferably, the polypeptides which comprise an amino acid sequence of which the sequence is at least 90% (in order of increasing preference at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7% or 99.8%) identical to the polypeptide of positions 31 to 490 according to SEQ ID no.1 (see sequence protocol).

More preferably, the polypeptides which comprise an amino acid sequence of which the sequence is at least 90% (in order of increasing preference at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7% or 99.8%) identical to the polypeptide of the positions 31 to 330 according to SEQ ID no.1 (cf. sequence protocol).

Polypeptides whose amino acid sequence is more than 99.0% identical to the sequence according to SEQ ID no.1 are particularly preferred.

A preferred mannanase enzyme is disclosed according to the claims of WO 99/64619, described in more detail in the description of this WO publication, and is therefore selected from at least one mannanase enzyme that is selected from at least one representative from the group that is formed from
i) Polypeptides which can be encoded by the part of the DNA sequence coding for the mannanase enzyme which is cloned into the plasmid present in Escherichia coli DSM 12197,
ii) Polypeptides comprising an amino acid sequence as shown at positions 33-340 of SEQ ID no.1 shown in WO 99/64619,
iii) Polypeptides comprising an amino acid sequence as shown at positions 31-990 or positions 91-1470, each from SEQ ID no.1 shown in WO 99/64619, or
iv) Analogues of the polypeptides defined in (i) or (ii) of which the sequence is at least 90% (and preferably in order of increasing preference at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8% or 99.9%) identical to the polypeptide, or are a fragment of (i), (ii) or (iii).

The sequence described in the context of the present invention under SEQ ID no.1 corresponds to the sequence disclosed in WO 99/64619 under SEQ ID no.2.

To interpret the features of the mannanase enzymes defined above and preferred according to the invention (vide supra) the entire disclosure of WO 99/64619 must also be expressly used in full. The mannanase enzymes mentioned as preferred in WO 99/64619 are also considered to be preferred in the context of the composition according to the invention.

Another preferred embodiment of the invention contains at least one mannanase from Paenibacillus sp. or one of the variants thereof which are described in WO 2017/079751 A1. Such a mannanase variant comprises an amino acid sequence which has at least two changes selected from
(i) at least one substitution at at least one of the positions selected from 1, 2, 3, 4, 6, 10, 19, 28, 30, 38, 59, 60, 61,62, 63, 66, 67, 68, 70, 71, 74, 75, 78, 80, 82, 93, 97, 103, 111, 124, 129, 131, 135, 136, 139, 143, 150, 167, 168, 184, 213, 214, 217, 225, 228, 235, 242, 244, 258, 259, 261, 283 and 284, and
(ii) an insertion at position 298, wherein said amino acid positions correspond to the positions according to SEQ ID no. 2 of this application. SEQ ID no.: 2 (corresponds to SEQ ID no. 14 of WO 2017/079751 A1).

To disclose the aforementioned preferred mannanase from Paenibacillus sp. or one of its variants, express and full reference is made to the disclosure of WO 2017/079751 A1. In particular, the definition for the letter code “Z” as an insertion or deletion in a parent or reference amino acid sequence also applies in this context to the variants. For an insertion relative to a stem or reference sequence, the code “Z” is noted on the left side of a position number and further comprises a number (e.g., .01) which is positioned in front of the code of the inserted amino acid to indicate the order of the insertion. For example, the insertion of the amino acid glutamine (Q) at position 298 is represented as “Z298.01 Q”; the insertion of amino acid X (where Xis any amino acid) at position 298 is represented as “Z298.01X”; and the insertion of three amino acids alanine (A), serine (S) and tyrosine (Y) between position 87 and 88 is represented as “Z87.01 N/Z87.02S/Z87.03Y”. For a deletion, the code “Z” is placed on the right side of the position number. For example, the deletion of alanine (A) from position 100 is represented as A100Z. For example, a combination of some of the above insertions and deletions is represented as: “G87S/Z87.01 N/Z87.02S/Z87.03Y/A100Z.”

In a preferred embodiment, such a mannanase variant comprises at least two changes which are selected from
(i) at least one substitution selected from the group consisting of M1V, M1L, A2S, T3R, G4S, Y6E, N10T, N10S, P19E, G28A, G28S, S30T, T38E, S59D, S59V, L60Q, Y61W, T62E, K63R, K63L, L66V, N67D, A68S, K70R, N71D, N74E, N74S, V75L, Q78D, Q78H, K80T, I82M, K93R, N97D, N97L, V103I, E111D, E111S, 1124V, Y129M, T131A, T135L, A136L, D139M, K143Q, K143R, N150T, F167Y, P168A, P168S, Q184D, Q184L, N213A, K214I, A217P, G225C, G225P, T228V, Y235L, Q242L, K244L, S258D, G259P, N261Q, N261R, D283S, T284A and T284E, and
(ii) an insertion at position Z298.01Q, wherein said amino acid positions correspond to the positions according to SEQ ID no. 2.

In a further preferred embodiment, such a mannanase variant comprises one of the following combinations of changes: P19E-T38E-K63L-N71D-Y129M-Q184L-K244L-S258D-N261R; N67D-Y129M-P168S-Q184L-K244L-S258D-G259P; P19E-K63L-N67D-Q78D-K80T-N97D-Y129M-G225C-T228V-K244L; P19E-T38E-N67D-N97D-Y129M-P168S-Q184L-K244L-S258D-N261R; P19E-T38E-N67D-N71D-Q78D-K80T-N97D-Y129M-P168S-G225C-K244L-S258D-N261R; T38E-K63L-N71D-N97D-Y129M-Q184L-G225C-T228V-Q242L-K244L-S258D-N261R; P19E-K63L-N71D-N97D-Y129M-Q184L-G225C-K244L-S258D-G259P; N10T-T38E-S59V-L60Q-K63R-L66V-A68S-N74S-V75L-N97D-V103I-Y129M-F167Y-Q184L-A217P-G225C-Y235L-K244L-S258D-N261R-Z298.01 Q; P19E-T38E-N67D-N71D-N97D-Y129M-F167Y-Q184L-A217P-K244L-S258D-N261R; T38E-K63L-N67D-Q78D-K80T-N97D-Y129M-P168S-Q184L-K244L-S258D-N261R; P19E-T38E-N67D-Y129M-P168S-Q184L-K244L-S258D-N261R; P19E-N67D-N97D-Y129M-P168S-Q184L-K244L; P19E-T38E-K63L-N71D-Y129M-P168S-G225C-T228V-K244L-S258D-N261R; P19E-T38E-N67D-N97D-Q184L-A217P-G225C-T228V-Y235L-K244L-S258D-N261R; N10T-P19E-G28S-S30T-T38E-N67D-N71D-N97D-Y129M-P168S-Q184L-G225C-Y235L-K244L-S258D-N261R-Z298.01Q; P19E-T38E-S59V-L60Q-K63R-N67D-N97D-V103I-Y129M-K143Q-F167Y-Q184L-G225C-Y235L-K244L-S258D-N261R-Z298.01 Q; P19E-T38E-N67D-N71D-Q78D-K80T-N97D-Y129M-P168S-G225C-T228V-K244L-S258D-N261R-Z298.01Q; P19E-T38E-S59V-L60Q-K63L-K70R-N71D-Q78D-K80T-N97D-E111D-Y129M-Q184L-G225C-T228V-Y235L-K244L-S258D-N261R-Z298.01Q; N10T-T38E-K63L-N71D-N97D-V103I-Y129M-F167Y-Q184L-G225C-T228V-Y235L-K244L-S258D-N261R-Z298.01 Q; N10T-P19E-T38E-N67D-Q78D-K80T-N97D-129M-K143Q-Q184L-A217P-G225C-T228V-Y235L-K244L-S258D-N261R-Z298.01 Q; N10T-P19E-T38E-S59V-L60Q-K63L-N97D-V103I-Y129M-F167Y-Q184L-G225C-Y235L-K244L-S258D-N261R-Z298.01 Q; P19E-S30T-T38E-S59V-L60Q-K63R-N67D-N97D-V103I-Y129M-F167Y-Q184L-G225C-T228V-Y235L-K244L-S258D-N261R-Z298.01 Q; P19E-S30T-T38E-S59V-L60Q-K63R-N67D-Q78D-K80T-N97D-I124V-Y129M-K143Q-F167Y-Q184L-G225C-Y235L-K244L-S258D-N261R-Z298.01 Q; N10S-P19E-S30T-T38E-S59V-L60Q-K63L-N67D-Q78H-K80T-I82M-N97D-Y129M-K143Q-F167Y-Q184L-G225C-Y235L-K244L-S258D-N261R-Z298.01 Q; N10T-P19E-S30T-T38E-S59V-L60Q-K63R-N67D-N97D-Y129M-K143Q-P168S-Q184L-G225C-T228V-Y235L-K244L-S258D-N261R-Z298.01 Q; G4S-N10T-P19E-T38E-N67D-Q78D-K80T-N97D-Y129M-Q184L-G225C-T228V-Y235L-K244L-S258D-N261R-Z298.01 Q; N10T-P19E-S30T-T38E-S59V-L60Q-K63L-K7OR-N71D-Q78D-K80T-N97D-Y129M-T131A-F167Y-Q184L-G225C-Y235L-K244L-S258D-N261R-Z298.01Q; N10T-P19E-S30T-T38E-S59V-L60Q-K63L-K70R-N71D-Q78D-K80T-N97D-E111D-Y129M-P168S-Q184L-G225C-T228V-Y235L-K244L-S258D-N261R-Z298.01 Q; P19E-S30T-T38E-S59V-L60Q-K63R-N67D-N97D-Y129M-P168S-Q184L-K214I-G225C-Y235L-K244L-S258D-N261R-Z298.01 Q; N10T-P19E-S30T-T38E-S59V-L60Q-K63R-N67D-N97D-Y129M-K143Q-P168S-Q184L-G225P-T228V-Y235L-K244L-S258D-N261R-Z298.01 Q; M1V-P19E-S30T-T38E-T62E-N67D-N71D-Q78D-N97D-Y129M-K143R-F167Y-P168S-Q184L-G225C-Y235L-K244L-S258D-N261R-T284A-Z298.01 Q; Y6E-N10T-P19E-G28S-S30T-T38E-K63L-N67D-N71D-N97D-E111S-Y129M-S135L-P168S-Q184L-G225C-T228V-Y235L-K244L-S258D-N261Q-D283S-Z298.01 Q; N10T-P19E-S30T-T38E-S59V-L60Q-K63R-N67D-N71D-N97D-V103I-Y129M-K1430-PI68S-Q184L-G225P-T228V-Y235L-K244L-S258D-N261R-Z298.01 Q; A2S-P19E-G28S-S30T-T38E-K63R-N67D-N71D-N74E-K93R-N97D-Y129M-N150T-P168S-Q184L-N213A-G225C-Y235L-K244L-S258D-N261Q-Z298.01 Q; M1L-N10T-P19E-G28A-S30T-T38E-K63L-N67D-N71D-Q78D-N97D-Y129M-A136L-P168A-Q184L-N213A-G225C-Y235L-K244L-S258D-N261R-Z298.01 Q; P19E-T38E-S59V-K63R-N67D-N97D-V10I-Y129M-F167Y-Q184L-G225C-T228V-Y235L-K244L-S258D-N261R-Z298.01 Q; N10T-P19E-G28A-S30T-T38E-K63R-N67D-N97D-Y129M-Q184L-G225C-T228V-Y235L-K244L-S258D-N261R-Z298.01 Q; T3R-N10T-P19E-G28A-S30T-T38E-T62E-N67D-N71D-K93R-N97L-E111S-Y129M-D139M-P168S-Q184L-G225C-Y235L-K244L-S258D-N261Q-Z298.01 Q; N10T-P19E-G28A-S30T-T38E-S59D-N67D-A68S-N71D-K93R-N97D-Y129M-K143Q-P168S-Q184D-G225C-Y235L-K244L-S258D-N261R-T284E-Z298.01 Q; P19E-K63L-N71D-Y129M-P168S-Q184L-G225C-K244L; P19E-N67D-N71D-Q78D-K80T-N97D-Y129M-P168S-Q184L-K244L; P19E-T38E-N67D-Y129M-P168S-Q184L-T228V-K244L; P19E-T38E-N67D-Y129M-Q184L-K244L-S258D-N261R; P19E-K63L-N71D-Y129M-P168S-Q184L-K244L-S258D-N261R; P19E-T38E-K63L-N71D-Y129M-P168S-Q184L-K244L-S258D-G259P; K63L-N71D-Y129M-K143R-P168S-Q184L-G225C-T228V-K244L-S258D-G259P or P19E-T38E-K63L-N71D-Y129M-P168S-Q184L-K244L-S258D-N261R, wherein the said amino acid positions correspond to the positions according to SEQ ID No. 2.

In a particularly preferred embodiment, such a mannanase variant comprises an amino acid sequence as shown in one of SEQ ID no.: 13 or SEQ ID no. 46-91 in WO 2017/079751 A1. Reference is made expressly and in full to the disclosure of WO 2018/184767 A1.

Another possible preferred embodiment of the invention contains a mannanase from Bacillus hemicellulosilyticus or one of the variants thereof which are described in WO 2018/184767 A1. Such a mannanase variant comprises an amino acid sequence which is at least 75% (increasingly preferably at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%) sequence identity to the amino acid sequence according to SEQ ID no. 16 from WO 2018/184767 A1.

A possible embodiment of the invention can be a mannanase from Bacillus clausii or contain one of the variants thereof which are described in WO 2018/184767 A1. Such a mannanase variant comprises an amino acid sequence which has at least 93% (increasingly preferably at least 94%, 95%, 96%, 97%, 98% or 99%) sequence identity to the amino acid sequence according to SEQ ID no.12 from WO 2018/184767 A1.

A possible embodiment of the invention can be a mannanase from Virgibacillus soli or one of the variants thereof which are described in WO 2018/184767 A1. Such a mannanase variant comprises an amino acid sequence which has at least 79% (increasingly preferably at least 80%, 81%, 82%, 83%, 84%, 85%, 86%, 87%, 88%, 89%, 90%, 91%, 92%, 93%, 94%, 95%, 96%, 97%, 98% or 99%) sequence identity to the amino acid sequence according to SEQ ID no. 20 from WO 2018/184767 A1.

The compositions according to the invention preferably additionally contain at least one further active substance. Within the meaning of the present invention, active substances are in particular:
textile care products such as plasticizers, water and re-stain repellents and impregnating agents, bleach activators, enzymes, coloring dyes, silicone oils, anti-redeposition agents, optical brighteners, graying inhibitors, anti-shrink agents, anti-crease agents, dye transfer inhibitors, antimicrobial active ingredients, germicides, fungicides, antioxidants, antistatic agents, ironing aids, anti-swelling and anti-slip agents, UV absorbers, cationic polymers,
skin care products or
perfume (oil) or odorants.

At least one active substance is preferably selected from enzymes, optical brighteners, builders, solvents, anti-redeposition agents, dye transfer inhibitors, coloring dyes, preservatives, perfume or mixtures of at least two of the aforementioned active substances.

The composition may further contain an additional bleaching agent different from the catechol compound according to formula (I). In a preferred embodiment, substantially no further bleaching agent is contained.

It is preferred if the composition according to the invention additionally has at least one further enzyme, in particular selected from protease, lipase, amylase, cellulase, hemicellulase, tannase, xylanase, xanthanase, xyloglucanase, β-glucosidase, pectinase, carrageenase, perhydrolase, oxidase, oxidoreductase or mixtures therefrom (particularly preferably from protease, amylase, lipase, cellulase, pectate lyase or mixtures thereof). The at least one additional, further enzyme is different from mannanase.

The amylase(s) is/are preferably an α-amylase. The hemicellulase is preferably a β-glucanase, a pectinase, a pullulanase. The cellulase is preferably a cellulase mixture or a single-component cellulase, preferably or predominantly an endoglucanase and/or a cellobiohydrolase. The oxidoreductase is preferably an oxidase, in particular a choline-oxidase, or a perhydrolase.

It is preferred according to the invention if at least one protease is contained as the enzyme. Proteases are some of the technically most important enzymes. They are the longest established enzymes for washing and cleaning agents, and are contained in virtually all modern, effective washing and cleaning agents. They bring about the decomposition of protein-containing stains on the item to be cleaned. Of these, in turn, proteases of the subtilisin type (subtilases, subtilopeptidases, EC 3.4.21.62) are particularly important and are serine proteases due to the catalytically active amino acids. They act as non-specific endopeptidases and hydrolize any acid amide bonds that are inside peptides or proteins. Their optimum pH is usually in the distinctly alkaline range. An overview of this family is given, for example, in the article “Subtilases: Subtilisin-like Proteases” by R. Siezen, pages 75-95 in “Subtilisin enzymes,” edited by R. Bott and C. Betzel, New York, 1996. Subtilases are, naturally, formed from microorganisms. In particular, the subtilisins formed and secreted by Bacillus species are the most significant group of subtilases.

A protease is an enzyme that cleaves peptide bonds by hydrolysis. According to the invention, each of the enzymes from class EC 3.4 is included (including each of the thirteen subclasses which fall thereunder). The EC number corresponds to the Enzyme Nomenclature 1992 of the NC-IUBMB, Academic Press, San Diego, Calif., including supplements 1 to 5, published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650.

Subtilase designates a subgroup of serine proteases. Serine proteases or serine peptidases are a subgroup of proteases that have serine in the active center of the enzyme which forms a covalent adduct together with the substrate. Furthermore, the subtilases (and the serine proteases) are characterized in that they have two further amino acid residues in the active center in addition to said serine together with histidine and aspartame. The subtilases can be divided into 6 subclasses, specifically the subtilisin family, the thermitase family, the proteinase K family, the lantibiotic peptidase family, the kexin family and the pyrrolysine family. The proteases which are preferably excluded or preferably contained in reduced amounts as part of the compositions according to the invention are endopeptidases (EC 3.4.21).

According to the invention, “protease activity” is present if the enzyme has proteolytic activity (EC 3.4). Different types of protease activity are known. The three main types are: trypsin-like, where the amide substrate is cleaved following the amino acids Arg or Lys at P1; chymotrypsin-like, where cleavage takes place following one of the hydrophobic amino acids at P1; and elastase-like, where the amide substrate is cleaved following Ala at P1.

The protease activity can be determined by the method described in Surfactant, Volume 7 (1970), pp. 125-132. Accordingly, it is given in PU (protease units). The protease activity of an enzyme can be determined according to common standard methods, such as in particular using BSA as substrate (bovine albumin) and/or using the AAPF method.

Surprisingly, it was found that a protease of the type of alkaline protease from Bacillus lentus DSM 5483 or a protease sufficiently similar to this (based on the sequence identity) which has a plurality of these changes in combination is particularly suitable for use in the composition according to the invention and advantageously stabilized in an improved manner therein. Advantages of using this protease thus arise in particular with regard to wash performance and/or stability.

Examples of the subtilisin proteases preferably used in detergent and cleaning agents are the subtilisins BPN' and Carlsberg, the protease PB92, the subtilisins 147 and 309, the alkaline protease from Bacillus lentus, in particular from Bacillus lentus DSM 5483, the subtilisin DY and the enzymes thermitase, proteinase K and proteases TW3 and TW7, which belong to the subtilases but no longer to the subtilisins in the narrower sense, and variants of said proteases having an amino acid sequence that has been altered with respect to the starting protease. Proteases are altered, selectively or randomly, by methods known from the prior art, and are thereby optimized for use in washing and cleaning agents, for example. This includes point, deletion or insertion mutagenesis, or fusion with other proteins or protein parts. Appropriately optimized variants are therefore known for the majority of proteases known from the prior art.

In the international applications WO95/23221A1, WO92/21760A1 and WO2013/060621A1, variants of the alkaline protease are from Bacillus lentus DSM 5483 are disclosed which are suitable for use in detergents or cleaning agents. Furthermore, the international patent applications WO2011/032988A1 and WO2016/096714A1, as well as the European patent application EP3044302A1, disclose detergents and cleaning agents which include variants of the alkaline protease from Bacillus lentus DSM 5483.

The concentration of the protease in the composition is preferably from 0.001-0.1 wt. %, more preferably from 0.01 to 0.06 wt. %, based on active protein.

The compositions preferably additionally contain at least one cellulase. A cellulase is an enzyme. Synonymous terms can be used for cellulases, in particular endoglucanase, endo-1,4-beta-glucanase, carboxymethyl cellulase, endo-1,4-beta-D-glucanase beta-1,4-glucanase, beta-1,4-endoglucanhydrolase, celludextrinase or avicelase. Crucial for whether an enzyme is a cellulase within the meaning of the invention is its ability to hydrolize 1,4-β-D-glucosidic bonds in cellulose.

Cellulases (endoglucanases, EG) which can be packaged according to the invention comprise, for example, the fungal cellulase preparation which is rich in endoglucanase (EG) and the refinements thereof which are provided by Novozymes under the trade name Celluzyme®. The products Endolase® and Carezyme®, also available from Novozymes, are based on 50 kD-EG and 43 kD-EG, respectively, from Humicola insolens DSM 1800. Further commercial products from this company that can be used are Cellusoft®, Renozyme®, and Celluclean®. It is also possible to use cellulases, for example, which are available from AB Enzymes, Finland, under the trade names Ecostone® and Biotouch®, and which are, at least in part, based on 20 kD-EG from Melanocarpus. Further cellulases from AB Enzymes are Econase® and Ecopulp®. Further suitable cellulases are from Bacillus sp. CBS 670.93 and CBS 669.93, wherein the cellulase from Bacillus sp. CBS 670.93 is available from Danisco/Genencor under the trade name Puradax®. Further commercial products that can be used from Danisco/Genencor are “Genencor detergent cellulase L” and IndiAge®Neutra.

Variants of these enzymes that can be obtained by point mutations may also be used according to the invention. Particularly preferred cellulases are Thielavia terrestris cellulase variants which are disclosed in international patent specification WO 98/12307, cellulases from Melanocarpus, in particular Melanocarpus albomyces, which are disclosed in international patent specification WO 97/14804, cellulases of the EGIII type from Trichoderma reesei which are disclosed in the European patent application EP 1 305 432 or variants obtainable therefrom, in particular those which are disclosed in the European patent applications EP 1240525 and EP 1305432, and cellulases which are disclosed in the international patent specifications WO 1992006165, WO 96/29397 and WO 02/099091. Reference is therefore expressly made to each disclosure, and the disclosure content thereof in this regard is therefore expressly included in the present invention.

Particularly preferred compositions according to the invention are characterized in that they have at least one cellulase of a 20K-cellulase that can be obtained from Melanocarpus sp. or Myriococcum sp. or of such a cellulase that has a homology thereto of more than 80% (increasingly preferably of more than 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8% or 99.9%) as additional cellulase.

The 20K-cellulase which is obtained from Melanocarpus sp. or Myriococcum sp. is known from international patent application WO 97/14804. As described there, it has a molecular weight of approximately 20 kDa and has at least 80% of its maximum activity at 50° C. in the pH range of from 4 to 9, wherein almost 50% of the maximum activity remains at 10 pH. As also described there, it can be isolated from Melanocarpus albomyces and produced in genetically engineered Trichoderma reseei transformants. Within the meaning of the present invention, cellulases which have a homology of more than 80% (increasingly preferably of more than 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8% or 99.9%) to 20K cellulase can also be used.

K20 cellulase is preferably used in amounts such that a composition according to the invention has a cellulolytic activity of from 1 NCU/g to 500 NCU/g (can be determined by the hydrolysis of 1 wt. % carboxymethyl cellulose at 50° C. and neutral pH and determination of the reducing sugar released in the process using dinitrosalicylic acid, as described by M. J. Bailey et al. in Enzyme Microb. Technol. 3: 153 (1981); 1 NCU defines the amount of enzyme that produces reducing sugar in an amount which corresponds to 1 nmol glucose per second), in particular from 2 NCU/g to 400 NCU/g and particularly preferably from 6 NCU/g to 200 NCU/g. In addition, the composition according to the invention can optionally contain further cellulases.

A composition according to the invention preferably contains 0.001 mg to 0.5 mg, in particular 0.02 mg to 0.3 mg, of cellulolytic protein per gram of the entire composition. The protein concentration can be determined using known methods, for example the bicinchonic acid process (BCA method, Pierce Chemical Co., Rockford, Ill.) or the Biuret method (A. G. Gornall, C. S. Bardawill and M. M. David, J. Biol. Chem. 177, 751-766, 1948).

It is also particularly preferred according to the invention to use at least one further second cellulase which is different from the first cellulase in addition to the at least one first cellulase of a 20K-cellulase which can be obtained from Melanocarpus sp. or Myriococcum sp. or of such a cellulase which has a homology thereto of more than 80% (increasingly preferably of more than 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8% or 99.9%).

It is preferred according to the invention if the compositions according to the invention additionally contain at least one lipase. Preferred lipase enzymes according to the invention are selected from at least one enzyme of the group which is formed from triacylglycerol lipase (EC 3.1.1.3), lipoprotein lipase (EC 3.1.1.34) and monoglyceride lipase (EC 3.1.1.23).

Furthermore, the lipase preferably contained in a composition according to the invention is naturally present in a microorganism of the species Thermomyces lanuginosus or Rhizopus oryzae or Mucor javanicus or is derived by mutagenesis from the aforementioned lipases which are naturally present. The compositions according to the invention particularly preferably contain at least one lipase which is naturally present in a microorganism of the species Thermomyces lanuginosus or is derived by mutagenesis from the aforementioned lipases which are naturally present in Thermomyces lanuginosus.

In this context, naturally present means that the lipase is a separate enzyme of the microorganism. The lipase can thus be expressed in the microorganism by a nucleic acid sequence which is part of the chromosomal DNA of the microorganism in its wild-type form. The lipase or the nucleic acid sequence coding for it is therefore present in the wild-type form of the microorganism and/or can be isolated from the wild-type form of the microorganism. In contrast, a lipase which is not naturally present in the microorganism or the nucleic acid sequence coding for it would have been introduced into the microorganism in a targeted manner using genetic engineering methods such that the microorganism would have been enriched with the lipase or the nucleic acid sequence coding for it. However, a lipase which is naturally present in a microorganism of the species Thermomyces lanuginosus or Rhizopus oryzae or Mucor javanicus may have been produced recombinantly by a different organism.

The fungus Thermomyces lanuginosus (also known as Humicola lanuginosa) belongs to the class Eu ratio mycetes (subclass Eurotiomycetidae), therein to the order Eurotiales and therein to the family Trichocomaceae and the genus Thermomyces. The fungus Rhizopus oryzae belongs to the class Zygomycetes (subclass Incertae sedis), therein to the order Mucorales and therein again to the family Mucoraceae and the genus Rhizopus. The fungus Mucor javanicus also belongs to the class Zygomycetes (subclass Incertae sedis), therein to the order Mucorales and therein again to the family Mucoraceae, and then therein to the genus Mucor. The names Thermomyces lanuginosus, Rhizopus oryzae and Mucor javanicus are the biological species names within the relevant genus.

Preferred lipases according to the invention are the lipase enzymes available from Amano Pharmaceuticals under the names Lipase MAP10®, Lipase LE® and Lipase F® (also Lipase JV®). For example, Lipase F® is naturally present in Rhizopus oryzae. Lipase MAP10®, for example, is naturally present in Mucor javanicus.

Compositions of a very particularly preferred embodiment of the invention contain at least one lipase which is selected from at least one or more polypeptides having an amino acid sequence which is at least 90% (and increasingly preferably at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8%, 99.9%) identical to the wild-type lipase from the strain DSM 4109 Thermomyces lanuginosus. It is again preferred if, based on said wild-type lipase from strain DSM 4109, there is at least the amino acid mutation N233R.

Within the scope of a further embodiment, in particular the lipases which are derived from the wild-type lipase from strain DSM 4109 and are selected from at least one lipase enzyme according to the claims of the publication WO 00/60063 A1 can preferably be used according to the invention. Reference is expressly made in full to the disclosure in publication WO 00/60063 A1.

At least one lipase which is derived from the wild-type lipase from strain DSM 4109 and in which, based on said wild-type lipase, an electrically neutral or negatively charged amino acid is at least substituted by a positively charged amino acid is particularly preferably used in the compositions of the invention. The charge is determined in water at a pH of 10. Negative amino acids within the meaning of the invention are E, D, Y and C. Positively charged amino acids within the meaning of the invention are R, K and H, in particular R and K. Neutral amino acids within the meaning of the invention are G, A, V, L, I, P, F, W, S, T, M, N, Q and C, if C forms a disulfide bridge.

In the context of this embodiment of the invention, it is also preferred if, based on the wild-type lipase from strain DSM 4109, at least one of the following amino acid exchanges is present in positions D96L, T213R and/or N233R, particularly preferably T213R and N233R.

A highly preferred lipase is commercially available from Novozymes (Denmark) under the trade name Lipex® and can advantageously be used in the cleaning compositions according to the invention. Particular preference here is the lipase Lipex® 100 L (ex Novozymes A/S, Denmark). Preferred compositions are characterized in that, based on the total weight of the composition, said Lipase enzyme from Lipex® 100 L is contained in a total amount of from 0.01 to 1.0 wt. %, in particular 0.02 to 0.1 wt. %.

Particularly preferably, the composition according to the invention also contains at least one α-amylase, particularly preferably in addition to the preferred protease of the alkaline protease type from Bacillus lentus DSM 5483 or in addition to a protease which is sufficiently similar to this (based on the sequence identity) and has a plurality of these modifications in combination.

α-amylases (EC 3.2.1.1) hydrolyze internal α-1,4-glycosidic bonds of starch and starch-like polymers as enzymes. This α-amylase activity is measured, for example, according to the applications WO 97/03160 A1 and GB 1296839 in KNU (Kilo Novo Units). 1 KNU represents the amount of enzyme that hydrolyzes 5.25 g of starch (available from Merck, Darmstadt, Germany) per hour at 37° C., at a pH of 5.6 and in the presence of 0.0043 M calcium ions. An alternative activity determination method is the DNS method which is described for example in the application WO 02/10356 A2. The oligosaccharides, disaccharides and glucose units released by the enzyme during the starch hydrolysis are then detected by oxidizing the reducing ends with dinitrosalicylic acid (DNS). The activity is obtained in pmol reducing sugars (based on maltose) per min and ml; this results in activity values in TAU. The same enzyme can be determined using different methods, wherein it is possible for the respective conversion factors to vary depending on the enzyme and therefore having to be determined on the basis of a standard. It can be roughly calculated that 1 KNU corresponds to approx. 50 TAU. A further activity determination method is measuring using the Quick-Start® test kits from Abbott, Abbott Park, Ill., USA.

A preferred field of application of the compositions according to the invention is the cleaning of textiles. Because detergents and cleaning agents for textiles mainly have alkaline pH values, α-amylases, which are active in the alkaline medium, are in particular used for this purpose. These are produced and secreted by microorganisms, i.e. fungi or bacteria, especially those of the genera Aspergillus and Bacillus. Based on these natural enzymes, there is still an almost unmanageable abundance of variants which have been derived via mutagenesis and which have specific advantages depending on the field of application.

Examples of these are α-amylases from Bacillus licheniformis, from B. amyloliquefaciens and from B. stearothermophilus, as well as the refinements thereof that have been improved for use in detergents or cleaning agents. The enzyme from B. licheniformis is available from Novozymes under the name Termamyl® and from Genencor under the name Purastar® ST. Development products of this α-amylase are available from Novozymes under the trade names Duramyl® and Termamyl® ultra, from Genencor under the name Purastar® OxAm, and from Daiwa Seiko Inc., Tokyo, Japan, as Keistase®. The α-amylase from B. amyloliquefaciens is marketed by Novozymes under the name BAN®, and derived variants from the α-amylase from B. stearothermophilus are marketed under the names BSG® and Novamyl , also by Novozymes.

Examples for α-amylases from other organisms are the refinements of α-amylase from Aspergillus niger and A. oryzae that are available under the trade name Fungamyl from Novozymes. A further commercial product is, for example, the amylase LT®.

The prior art includes, inter alia, the three patent applications WO 96/23873 A1, WO 00/60060 A2 and WO 01/66712 A2, which have been registered by Novozymes. WO 96/23873 A1 describes a plurality of different point mutations in a total of more than 30 different positions in four different wild-type amylases and claims that for all amylases having at least 80% identity to one of those four; they should have modified enzymatic properties with regard to thermal stability, oxidation stability and calcium dependency. The application WO 00/60060 A2 also mentions a large number of possible amino acid exchanges in 10 different positions on the α-amylases from two different microorganisms and claims that for all amylases having a homology of at least 96% identity to them. Finally, WO 01/66712 A2 describes 31 different amino acid positions, some of which are identical to the above-mentioned positions, that have been mutated into one of the two α-amylases which are cited in the application WO 00/60060 A2.

WO 96/23873 A1, for example, specifically gives the possibility of replacing an M in position 9 in the aforementioned α-amylases with an L according to the counting of AA560, M in position 202 with L and deleting the amino acids in positions 182 and 183 (or 183 and 184). WO 00/60060 A2 specifically discloses, inter alia, the amino acid variation N195X (i.e. in principle with any other amino acid). WO 01/66712 A2 discloses, inter alia, the amino acid variations R118K, G186X (including in particular the G186R exchange which is not relevant here), N299X (including in particular the N299A exchange which is not relevant here), R320K, E345R and R458K.

In addition to the preferred protease of the alkaline protease type from Bacillus lentus DSM 5483 or a protease which is sufficiently similar to this (based on the sequence identity) which has several of these modifications in combination, the composition according to the invention very particularly preferably also contains at least one α-amylase which has a higher activity at temperatures between 10 and 20° C. than the amylase having the trade name “Stainzyme 12 L” from Novozymes.

Detergents that are preferred according to the invention contain α-amylase in a total amount of from 0.01 to 1.0 wt. %, in particular 0.02 to 0.1 wt. %.

It is preferred that at least one optical brightener is selected from the substance classes of distyrylbiphenyls, stilbenes, 4,4′-diamino-2,2′-stilbene disulfonic acids, cumarines, dihydroquinolones, 1,3-diarylpyrazolines, naphthalic acid imides, benzoxazole systems, benzisoxazole systems, benzimidazole systems, pyrene derivatives substituted with heterocycles, and mixtures thereof. These substance classes of optical brighteners have a high stability, a high light and oxygen resistance and a high affinity for fibers.

The following optical brighteners, which are selected from the group consisting of disodium-4,4 ′-bis-(2-morpholino-4-anilino-s-triazin-6-ylamino)stilbene disulfonate, disodium-2,2′-bis-(phenyl-styryl) disulfonate, 4,4′-bis[(4-anilino-6-[bis(2-hydroxyethyl)amino]-1,3,5-triazin-2-yl)amino]stilben-2,2′-disulfonic acid, hexasodium-2,2′-[vinylenbis[(3-sulphonato-4,1-phenylen)imino[6-(diethylamino)-1,3,5-triazin-4,2-diyl]imino]]bis-(benzol-1,4-disulfonate), 2,2′-(2,5-thiophendiyl)bis[5-1,1-dimethylethyl)-benzoxazole (available, for example, as Tinopal® SFP from BASF SE) and/or 2,5-bis(benzoxazol-2-yl)thiophene, can be incorporated particularly well and in a stable manner.

According to the invention, the composition can furthermore comprise builders. Polymeric polycarboxylates are suitable as builders, for example. These are, for example, the alkali metal salts of polyacrylic acid or of polymethacrylic acid, for example those having a relative molecular mass of from 600 to 750,000 g/mol.

Suitable polymers are in particular polyacrylates which preferably have a molecular mass of from 1,000 to 15,000 g/mol. Due to their superior solubility, the short-chain polyacrylates, which have molar masses of from 1,000 to 10,000 g/mol, and particularly preferably from 1,000 to 5,000 g/mol, can in turn be preferred from this group.

In addition, copolymeric polycarboxylates are suitable, in particular those of acrylic acid with methacrylic acid and of acrylic acid or methacrylic acid with maleic acid. To improve water solubility, the polymers can also contain allyl sulfonic acids, such as allyloxybenzene sulfonic acid and methallyl sulfonic acid, as monomers.

Suitable builders that can be contained in the composition according to the invention are in particular also silicates, aluminum silicates (in particular zeolites), carbonates, salts of organic di- and polycarboxylic acids, and mixtures of these substances. Organic builders are particularly suitable as additional builders, for example the polycarboxylic acids which can be used in the form of the sodium salts thereof or as acids, wherein polycarboxylic acids are understood to mean those carboxylic acids that carry more than one acid function. These include, for example, adipic acid, succinic acid, glutaric acid, malic acid, tartaric acid, maleic acid, fumaric acid, saccharic acids, aminocarboxylic acids, in particular glutamic acid-N,N-diacetic acid (GLDA) and methylglycine-N,N-diacetic acid (MGDA), and mixtures thereof. Polymeric polycarboxylates are also suitable as builders. These are, for example, the alkali metal salts of polyacrylic acid or of polymethacrylic acid, for example those having a relative molecular mass of from 600 to 750,000 g/mol. Suitable polymers are in particular polyacrylates which preferably have a molecular mass of from 1,000 to 15,000 g/mol. Due to their superior solubility, the short-chain polyacrylates, which have molar masses of from 1,000 to 10,000 g/mol, and particularly preferably from 1,000 to 5,000 g/mol, can in turn be preferred from this group. In addition, copolymeric polycarboxylates are suitable, in particular those of acrylic acid with methacrylic acid and of acrylic acid or methacrylic acid with maleic acid. To improve water solubility, the polymers can also contain allyl sulfonic acids, such as allyloxybenzene sulfonic acid and methallyl sulfonic acid, as monomers. Soluble builders, such as acrylic polymers having a molar mass of from 1,000 to 5,000 g/mol, are preferably used in liquid components.

However, soluble builders, such as citric acid, or acrylic polymers having a molar mass of from 1,000 to 5,000 g/mol are particularly preferably used.

The composition preferably also contains one or more non-aqueous solvents. Suitable non-aqueous solvents comprise mono- or polyhydric alcohols or glycol ethers, such as ethanol, n-propanol, i-propanol, butanols, glycol, propanediol, butanediol, methylpropanediol, glycerol, glycols, such as diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxytriglycol, ethoxytriglycol, butoxytriglycol, 1-butoxyethoxy-2-propanol, 3-methyl-3-methoxybutanol, 2,2-dimethyl-4-hydroxymethyl-1,3-dioxolane, propylene-glycol-t-butylether, di-n-octylether, and low-molecular polyalkylene glycols, such as PEG 400, and mixtures of these solvents.

Preferably, the solvents are selected from ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxytriglycol, ethoxytriglycol, butoxytriglycol, 1-butoxyethoxy-2-propanol, 3-methyl-3-methoxybutanol, propylene-glycol-t-butylether, di-n-octylether, and mixtures of these solvents.

The compositions according to the invention preferably contain at least one tinting dye as a further active ingredient. Tinting dyes per se are known to the person skilled in the art. All tinting dyes are useful in the present invention. It is preferred to use tinting dyes which are essentially free of transition metals.

Particularly preferred are the tinting dyes methine blue and violet dyes, azo dyes, azine dyes, oxazine dyes, and xanthene dyes or dyes based on anthraquinone, acridine, benzodifuran, benzodifuranone, carotenoids, coumarin, cyanines, diazahemicyanines, diphenyl hemigomethane, diphenylmethane, formazanine, hemicyanides, indigoids, methine, napthalamides, naphthoquinones, nitro and nitroso compounds, oxazines, pyrazoles, polyethyleneimines, stilbene, styrene, triarylmethanes, triphenylmethanes, xanthenes, triphenooxazines, and thiophenes or mixtures thereof.

Suitable tinting dyes are disclosed for example in WO 2006/004876 A1, WO 2014/089386 A1, WO 2015/110291 A1, WO2015/042209 A1, WO 2012/059363 A1, EP 2852639 A1, EP 2 440645 A1, EP 2 864 464 Al and WO 2006/055787 A1.

In preferred aspects, at least one tinting dye is selected from compounds with the general formula H-1:

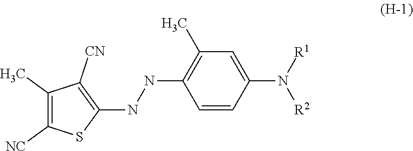

where R¹ and R² are independently selected from [(CH₂CR′HO)ₓ(CH₂CR″HO)_{y}Q], C1-12 alkyl, C6-10 aryl, C7-22 arylalkyl, with the proviso that at least one of R¹ and R² is [(CH₂CR′HO)ₓ(CH₂CR″HO)_{y}Q], wherein R′ is selected from the group consisting of H, C1-4 alkyl, CH2O(CH₂CH₂O)_{z}Q, phenyl and —CH₂OR⁵; wherein R″ is selected from H, C1-4 alkyl, CH₂O(CH₂CH201)_{z}Q, phenyl and —CH₂OR⁵; wherein 1 or 2≤x+y≤50, preferably x+y≤25, more preferably x+y≤10; wherein y >; wherein z=0 or 1 to 20, preferably 0 or 10 or 5; and wherein Q is selected from the group consisting of H and Y and wherein Y is as defined below; with the proviso that the dye comprises at least one radical Q which is Y; each R⁵ is selected from the group consisting of C1-16 linear or branched alkyl, C6-14 aryl, and C7-16 arylalkyl; R⁵ is preferably selected from the group consisting of methyl, ethyl, propyl, isopropyl, butyl, sec-butyl, isobutyl, tert-butyl, hexyl, 2-ethylhexyl, octyl, decyl, dodecyl, tetradecyl, hexadecyl, phenyl, benzyl, 2-phenylethyl, naphthyl, and mixtures thereof; and where Y is an organic radical represented by Formula H-2:

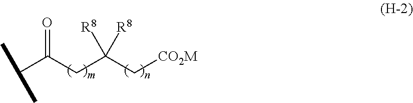

where, independently of one another, each radical is Y, M or H or a charge-balancing cation; m is 0 to 5, preferably 0, 1, 2 or 3; n is 0 to 5, preferably 0, 1, 2 or 3; the sum of m+n is 1 to 10, preferably 1, 2 or 3; each R⁸ is independently selected from the group consisting of H and C3-18 or C4-18 or even C4-7 and/or C9-18 alkenyl and wherein at least one radical R⁸ is not H.

A preferred anthraquinone-based tinting dye is a compound according to formula (H-3):

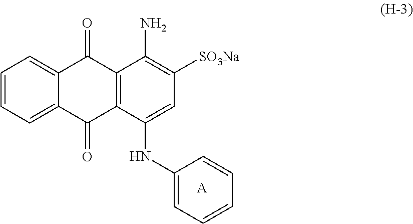

wherein if the ring A is substituted by a reactive radical, this radical is preferably selected from the group selected from: dichlorotriazinyl, difluorochloropyrimidine, monofluorotrazinyl, monofluorochlorotrazinyl, dichloroquinoxaline, difluorotriazine, monochlorotriazinyl, trichloropyrimidine-2-bromoprop-2-enamide; 2,3-dibromopropanamide; and sulfooxyethyl sulfonyl. The ring A can additionally be substituted by an organic radical selected from C1-8 alkyl or SO₃Na.

Another preferred anthraquinone-based tinting dye is a compound according to formula (H-4):

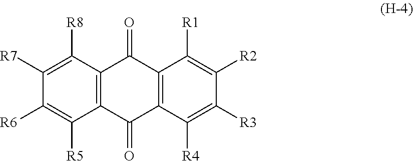

wherein R1, R4, R5 and R8 are independently selected from the radicals consisting of —H, —OH, —NH₂, NHCOCH₃ and —NO₂, with the proviso that at most one radical is an —NO₂ radical and at most two —H radicals are present as R1, R4, R5 and R8; and R2, R3, R6 and R7 are selected from —H, F, Br, CI or —NO2 and —O-aryl. 1-hydroxy-4-(p-tolylamino)antraquinone, 1-hydroxy-4-[(4-methylphenyl)amino]-9, 10-anthracenedione and 1-hydroxy-4-(4-methylanilino) anthraquinone are particularly preferred.

Monoazo dyes of the formula (H-5) are also preferred:

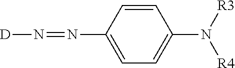

wherein R3 and R4 are optionally substituted C2 to C12 alkyls which optionally contain an ether (-0-) or ester bridge in the chain, and the chain is optionally substituted with —CI, —Br, —CN, —NO₂, and —SO₂CH₃,and D stands for an aromatic or heteroaromatic radical, which are optionally substituted with —Cl, —Br, —CN, —NO₂, —SO₂CH₃ and —NHCOR, wherein R is selected from—CH₃, —C₂H₅ and —CH₂CI.

Compounds of the formulas H-6 and H-7 are particularly preferred:

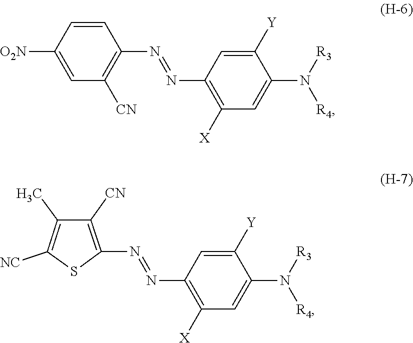

wherein X and Y are selected from —Cl, —Br, —CN, —NO₂, —SO₂CH₃ and —NHCOR, wherein R is selected from —CH₃, —C₂H₅ and —CH₂CI. Preferably X is NHCOCH₃ or NHCOCH₂CI.

Further preferred tinting dyes have the formula H-8:

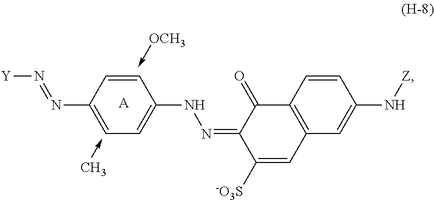

wherein Z is H or phenyl, the ring A is preferably substituted by a methyl or methoxy radical at the positions indicated by the arrows, where the ring A can also be a naphthyl ring, the radical Y is a phenyl or naphthyl ring, which is substituted by a sulfate radical and can be mono- or disubstituted by two methyl radicals. The tinting dyes according to formula H-9 are also preferred:

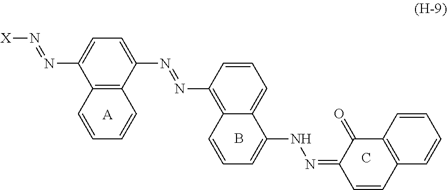

wherein at least two of the naphthyl rings A, B and C are substituted by a sulphonate radical, the ring C at the 5-position can be substituted by an NH₂ or NHPh radical, X is a phenyl or naphthyl ring which is substituted with up to 2 sulfonate radicals and which can be substituted at the 2-position with an —OH radical, and which can also be substituted with an NH₂ or NHPh radical.

Furthermore, at least one tinting dye can have the following formula H-10:

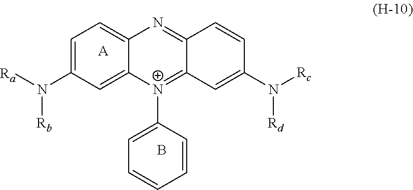

wherein Rₐ, R_{b}, R_{c} and R_{d} are selected from: H, a branched or linear C1-C7 alkyl radical, benzyl, phenyl, and naphthyl, wherein the dye having at least one radical —SO₃ or —COO⁻, preferably two —SO₃″ radicals, is substituted; ring B has no negatively charged radicals or salts thereof; and ring A may be substituted to form naphthyl; wherein the dye can optionally be further substituted by radicals selected from: amine, methyl, ethyl, hydroxyl, methoxy, ethoxy, phenoxy, Cl, Br, I, F and NO₂.

Further preferred tinting dyes have the formula H-11:

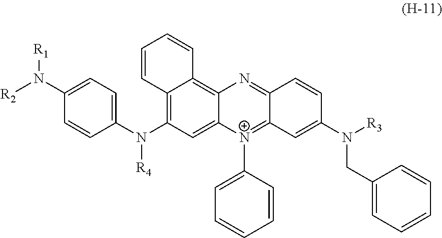

wherein R1, R2, R3 and R4 are selected from the group consisting of: H, Me, Et, n-Pr and i-Pr; and the dye is optionally substituted by a methoxy radical.

Further preferred tinting dyes have the formula H-12:

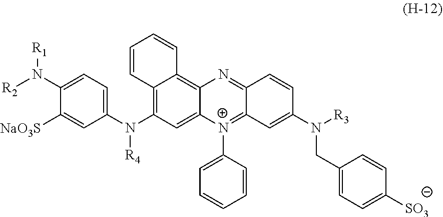

wherein R1, R2, R3 and R4 are selected from the group consisting of: H, Me, Et, n-Pr and i-Pr; and the dye is optionally substituted by a methoxy radical.

Furthermore, tinting dyes represented by the formula H-13 are preferred

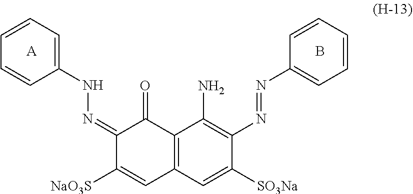

wherein one or both rings A and B are substituted with a reactive radical, preferably with -—SO₃Na, and optionally also —CH₃, —C₂H₅ and —OCH₃.

Further preferred tinting dyes have the formula H-14:

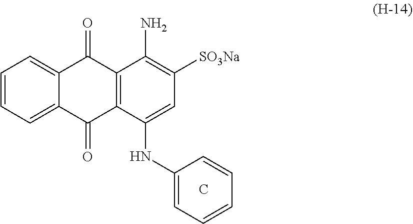

wherein ring C is substituted with a reactive radical, preferably with —SO₃Na, and optionally also —CH₃, —C₂H₅ and —OCH₃.

Also preferred tinting dyes have the formula H-15:

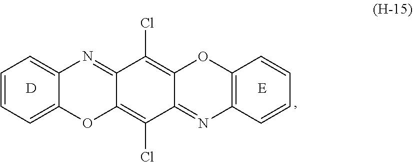

wherein rings D and E are substituted with a reactive radical, preferably with —SO₃Na.

Exemplary preferred compounds for a tinting dye which can be used according to the invention are

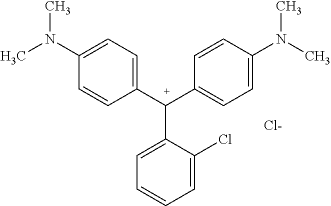

also known as Basic Blue 1,

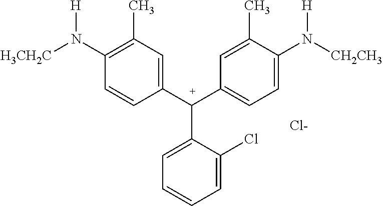

also known as Basic Blue 5,

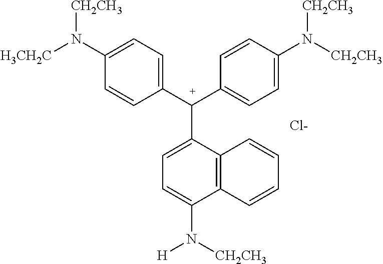

also known as Basic Blue 7,

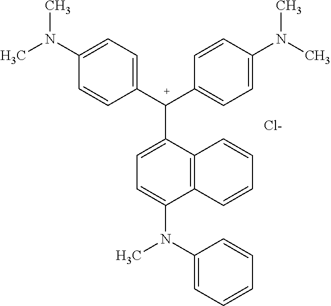

also known as Basic Blue 8,

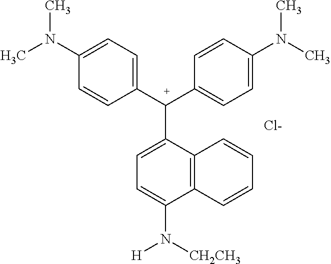

also known as Basic Blue 11,

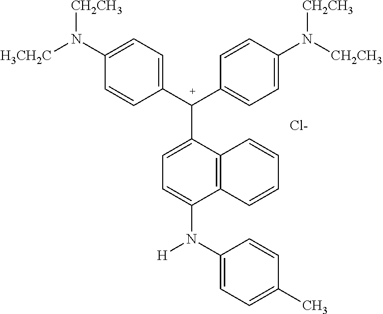

also known as Basic Blue 15,

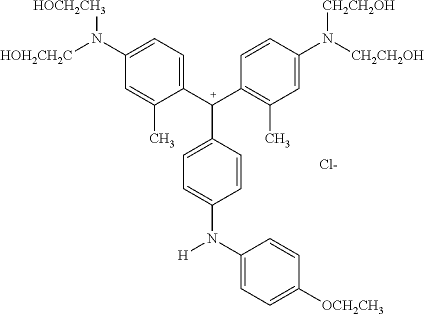

also known as Basic Blue 18,

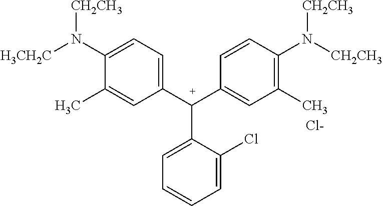

also known as Basic Blue 23,

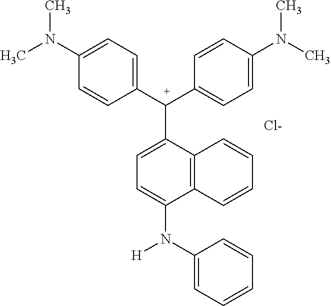

also known as Basic Blue 26,

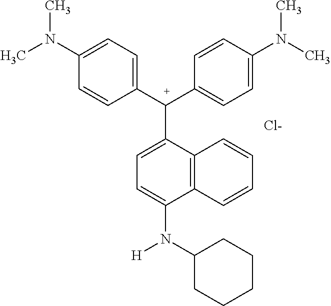

also known as Basic Blue 55,

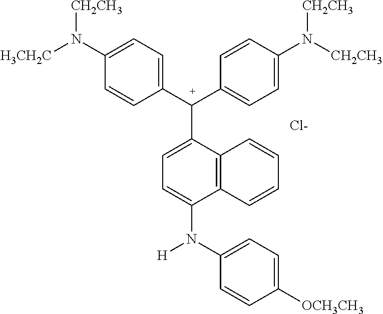

also known as Basic Blue 81,

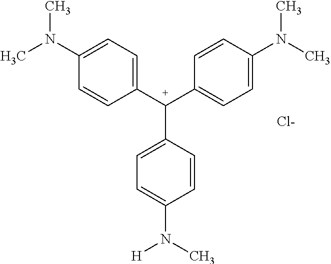

also known as Basic Violet 1,

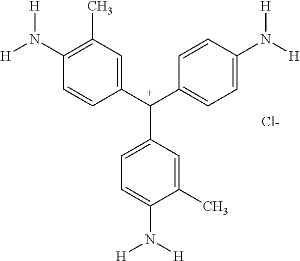 [SMILES: Cc1cc([C+](c2ccc(N)cc2)c2ccc(N)c(C)c2)ccc1N ; [Cl-]]

also known as Basic Violet 2,

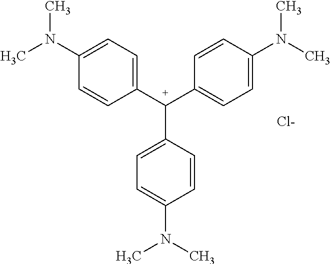

also known as Basic Violet 3,

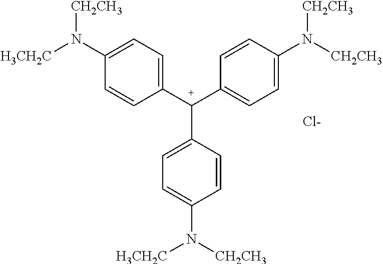

also known as Basic Violet 4,

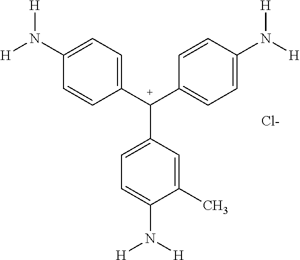 [SMILES: Cc1cc([C+](c2ccc(N)cc2)c2ccc(N)cc2)ccc1N ; [Cl-]]

also known as Basic Violet 14,

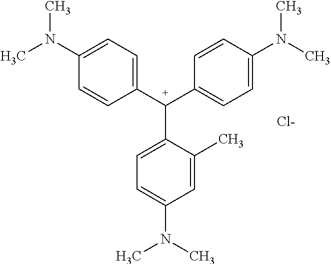

also known as Basic Violet 23,

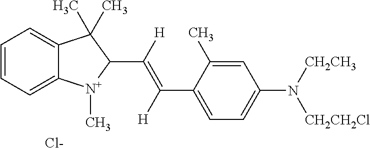

also known as Basic Violet 7,

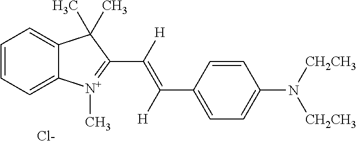 [SMILES: CCN(CC)c1ccc(/C=C/C2=[N+](C)c3ccccc3C2(C)C)cc1 ; [Cl-]]

also known as Basic Violet 16,

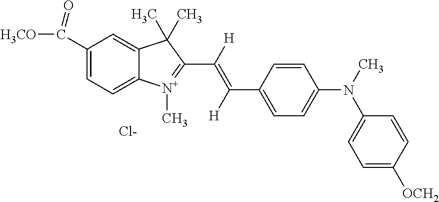

also known as Basic Violet 21,

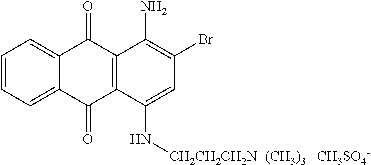

also known as Basic Violet 21,

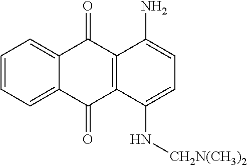

also known as Basic Violet 47,

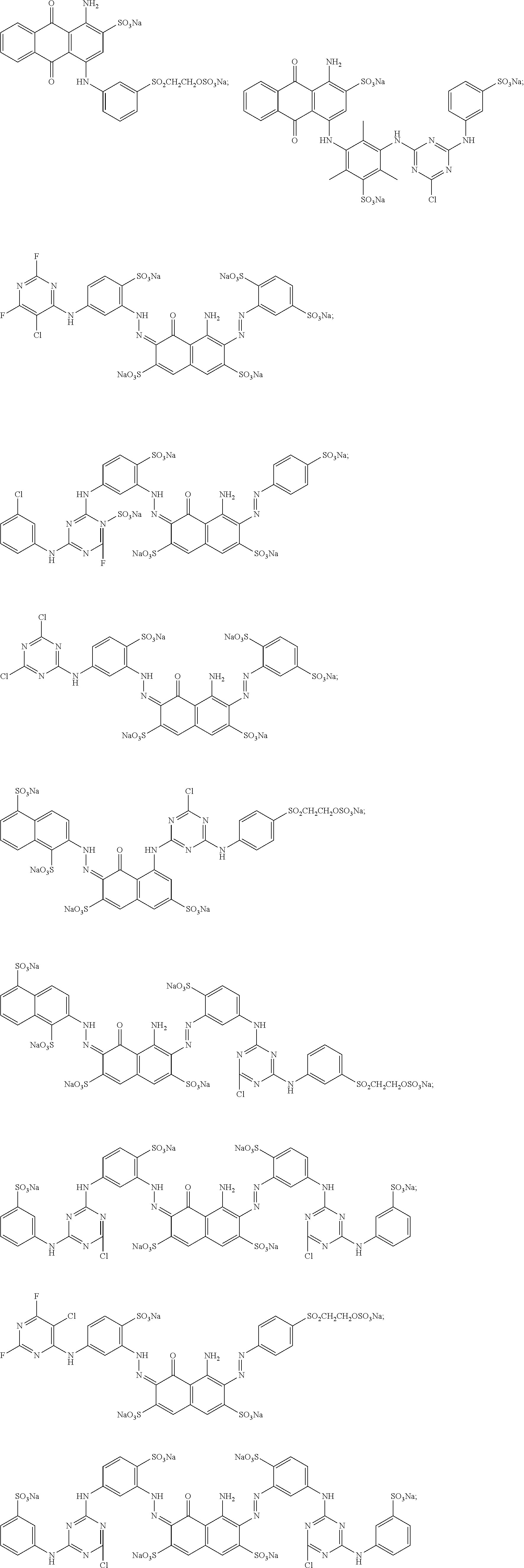

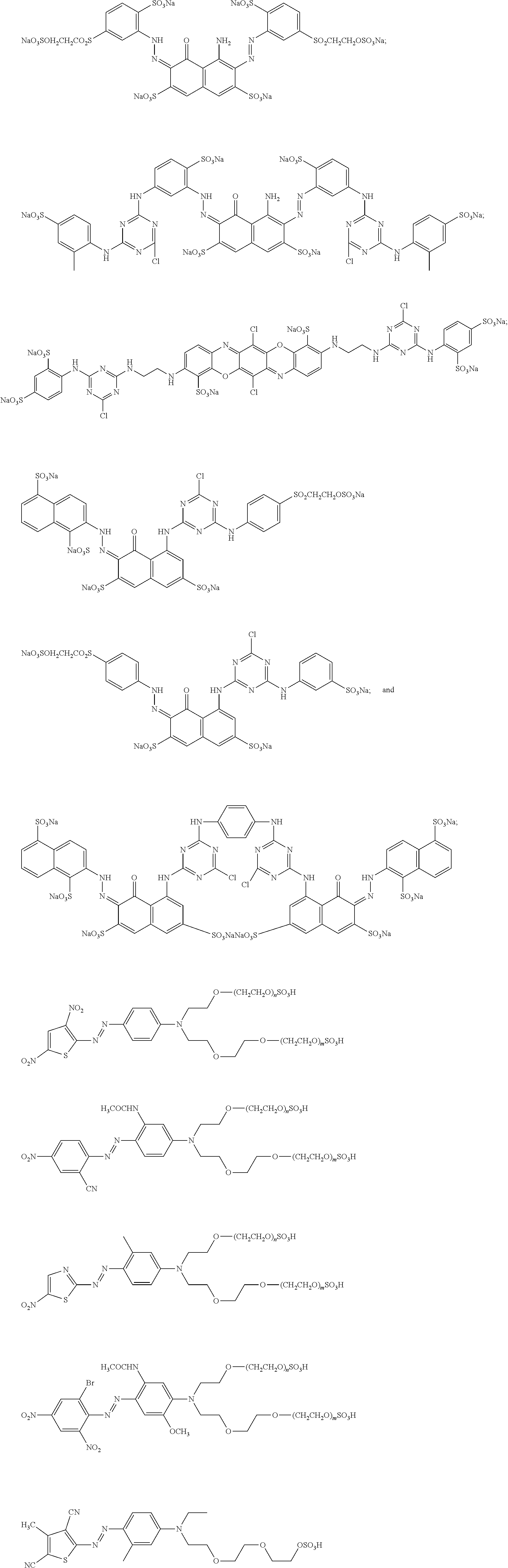

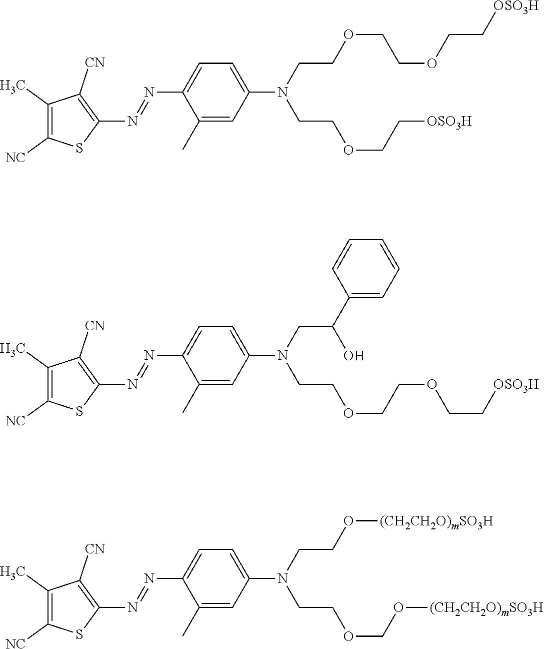

wherein in the above five compounds, n is an integer from 0 to 7 and m is an integer from 0 to 7.

Additionally, the compositions according to the invention can also contain components which positively influence the capability for washing out oil and grease from textiles, or what are referred to as soil-release active ingredients. This effect is particularly apparent when a textile is soiled which has previously been washed several times using an agent which contains this deoiling and degreasing component. Preferred deoiling and degreasing components include, for example, non-ionic cellulose ethers such as methylcellulose and methyl hydroxypropyl cellulose having a proportion of from 15 to 30 wt. % of methoxyl groups and from 1 to 15 wt. % of hydroxypropoxyl groups, in each case based on the non-ionic cellulose ether, and the polymers of phthalic acid and/or terephthalic acid known from the prior art, or derivatives thereof, with monomeric and/or polymeric diols, in particular polymers of ethylene terephthalates and/or polyethylene glycol terephthalates or anionically and/or non-ionically modified derivatives thereof. These are commercially available, for example, under the trade name Texcare®.

Anti-redeposition agents can be used in particular on (co)polymers based on polyethyleneimine, polyvinyl acetate and polyethylene glycol, preferably in mixtures with anti-redeposition agents.

The composition can preferably also contain dye transfer inhibitors, preferably in amounts of from 0.1 wt. % to 2 wt. %, in particular from 0.1 wt. % to 1 wt. %, which, in a preferred embodiment of the invention, are polymers of vinylpyrrolidone, vinyl imidazole or vinyl pyridine-N-oxide or copolymers thereof.

The function of graying inhibitors is to keep the dirt that is removed from the textile fibers suspended in the liquor. Water-soluble colloids, which are usually organic, are suitable for this purpose, for example starch, sizing material, gelatin, salts of ethercarboxylic acids or ethersulfonic acids of starch or of cellulose, or salts of acidic sulfuric acid esters of cellulose or of starch. Water-soluble polyamides containing acid groups are also suitable for this purpose. Starch derivatives other than those mentioned above may also be used, for example aldehyde starches. Cellulose ethers, such as carboxymethyl cellulose (sodium salt), methyl cellulose, hydroxyalkyl cellulose, and mixed ethers, such as methyl hydroxyethyl cellulose, methyl hydroxypropyl cellulose, methyl carboxymethyl cellulose and mixtures thereof, can preferably be used, for example, in amounts of from 0.1 to 5 wt. %, based on the composition.

It is preferred for the dye transfer inhibitor to be a polymer or a copolymer of cyclic amines such as vinylpyrrolidone and/or vinylimidazole. Polymers suitable as a dye transfer inhibitor include polyvinylpyrrolidone (PVP), polyvinylimidazole (PVI), copolymers of vinylpyrrolidone and vinylimidazole (PVP/PVI), polyvinylpyridine-N-oxide, poly-N-carboxymethyl-4-vinylpyridium chloride, polyethylene glycol-modified copolymers of vinylpyrrolidone and vinylimidazole, and mixtures thereof. Particularly preferably, polyvinylpyrrolidone (PVP), polyvinylimidazole (PVI) or copolymers of vinylpyrrolidone and vinylimidazole (PVP/PVI) are used as dye transfer inhibitors. The polyvinylpyrrolidones (PVP) used preferably have an average molecular weight of from 2,500 to 400,000 and are commercially available from ISP Chemicals as PVP K 15, PVP K 30, PVP K 60 or PVP K 90, or from BASF as Sokalan® HP 50 or Sokalan® HP 53. The copolymers of vinylpyrrolidone and vinylimidazole (PVP/PVI) used preferably have a molecular weight in the range of from 5,000 to 100,000 g/mol. A PVP/PVI copolymer is commercially available from BASF under the name Sokalan® HP 56, for example. Other dye transfer inhibitors that can be extremely preferably used are polyethylene glycol-modified copolymers of vinylpyrrolidone and vinylimidazole, which for example are available from BASF under the name Sokalan® HP 66.

The composition is used in particular in the context of a washing process for textiles. The compositions described herein are suitable as washing aids which are used as textile pre-treatment and post-treatment agents in textile washing, i.e. as agents with which the item of laundry is brought into contact before the actual washing, for example in order to dissolve stubborn stains.

The composition can be in a portion prepared for a wash cycle (e.g. as a water-soluble multi-chamber pouch) or in a multi-chamber storage container with a possible mixing chamber.

Within the meaning of the invention, a surfactant-containing liquor is a liquid preparation for treating a substrate that can be obtained by using the multi-component detergent of the present invention by diluting it with at least one solvent (preferably water). Fabrics or textiles (such as clothing) can be used as substrates. The compositions according to the invention are preferably used to provide a surfactant-containing liquor for automatic cleaning methods, as they are carried out, for example, by a washing machine for textiles.

If it is a multi-component detergent, the further components of the multi-component detergent can be in any administration form established according to the prior art and/or in any expedient administration form. These include, for example, liquid, gel-like or pasty dosage forms. The container is preferably a pouch having two, three, four, five, six, seven or eight chambers, packaged both in large containers and in portions.

However, it is very particularly preferred according to the invention if the multi-component detergent comprises only liquid components, wherein the liquid components preferably are packaged separately from one another by a wrapping.

In another particularly preferred embodiment, the multi-component detergent is a multi-component color detergent, in particular a liquid detergent, i.e. a textile detergent for colored textiles.

The detergents according to the invention preferably contain at least one alkalizing agent or the salt thereof in a total amount of from 1 to 20 wt. %, preferably 2 to 15 wt. %.

The total amount of the alkalizing agent and the salt thereof, or the total amount of all of the following preferred representatives, is calculated on the basis of the base form, i.e. if the alkalizing agent in the detergent according to the invention is (partly) in its salt form, the counterion is neglected in the amount calculation and only the base form without the absorbed proton is assumed for the salt portion.

The alkalizing agents are preferably selected from (C₂ to C₆) alkanolamine, sodium hydroxide, potassium hydroxide, sodium carbonate, sodium hydrogen carbonate or mixtures thereof.

According to the invention, the term (C₂ to C₆) alkanolamine is understood to mean organic amine compounds which have a carbon skeleton of two to six carbon atoms to which at least one amino group (preferably exactly one amino group) and at least one hydroxyl group (again preferably exactly one hydroxyl group) binds.

Preferred (C₂ to C₆) alkanolamines according to the invention are primary amines.

In the context of the invention, it is preferred that at least one (C₂ to C₆) alkanolamine having exactly one amino group is used. It is also preferably a primary amine.

The composition according to the invention preferably contains at least one (C₂ to C₆)-alkanolamine selected from 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan 4-ol, 3-amino-2-methylpropan-1-ol, 1-amino-2-methylpropan-2-ol, 3-aminopropane-1,2-diol, 2-amino-2-methylpropan-1,3-diol (especially from 2-aminoethan-1-ol, 2-amino-2-methylpropan-1-ol, 2-amino-2-methyl-propan-1,3-diol), or mixtures thereof. Monoethanolamine has proven to be a very particularly suitable (C₂ to C₆) alkanolamine as an alkalizing agent.

(C₂ to C₆) alkanolamine or the salt thereof is particularly preferably contained in a total amount of from 1 to 20 wt. %, preferably 2 to 15 wt. %, in the detergents according to the invention, in each case based on the basic form.

According to the invention, the liquid composition is preferably located in a container (pouch) consisting of water-soluble material. The container for said multi-component detergent comprises at least two spatially separate chambers (multi-chamber pouch), for example 2, 3, 4, 5, 6, 7 or 8 chambers. These chambers are separated from one another in such a way that the liquid or liquid and solid components of the detergent contained therein do not come into contact with one another. They can be separated, for example, by a wall made of the same material as the container itself.

It is therefore particularly preferred according to the invention to package the multi-component detergent in a container consisting of water-soluble material with at least two separate chambers.

A material is water-soluble within the meaning of the present invention if 0.1 g of the material dissolves in 800 mL water at 20° C. when stirred (stirring speed of magnetic stirrer 300 rpm, stirring rod: 6.8 cm long, diameter 10 mm, beaker 1000 mL low form from Schott, Mainz) within 600 seconds such that single solid particles of the material are no longer visible to the naked eye.

The water solubility of the material used for producing pouches for the wrapping can be determined by means of a square film of said material (film: 22×22 mm having a thickness of 76 μm) fixed in a square frame (edge length on the inside: 20 mm) according to the following measurement protocol. Said framed film is submerged into 800 ml of distilled water, temperature-controlled to 20° C., in a 1-liter beaker with a circular base (Schott, Mainz, beaker glass 1,000 mL, low form), so that the surface of the tensioned film is arranged at a right angle to the base of the beaker, the upper edge of the frame is 1 cm below the water surface, and the lower edge of the frame is oriented in parallel with the base of the beaker such that the lower edge of the frame extends along the radius of the base of the beaker and the center of the lower edge of the frame is arranged above the center of the radius of the beaker bottom. The material should dissolve within 600 seconds when stirred (stirring speed of magnetic stirrer 300 rpm, stirring rod: 6.8 cm long, diameter 10 mm), such that single solid film particles are no longer visible to the naked eye.

The water-soluble or water-dispersible material can comprise a polymer, a copolymer or mixtures thereof. Water-soluble polymers within the meaning of the invention are polymers which are soluble in water at more than 2.5 wt. % at room temperature.

Preferred water-soluble materials preferably comprise at least partly at least one substance from the group consisting of (acetalized) polyvinyl alcohol, polyvinyl pyrrolidone, polyethylene oxide, gelatin, polyvinyl alcohols substituted with sulphate, carbonate and/or citrate, polyalkylene oxides, acrylamides, cellulose esters, cellulose ethers, cellulose amides, cellulose, polyvinyl acetates, polycarboxylic acids and the salts thereof, polyamino acids or peptides, polyamides, polyacrylamides, copolymers of maleic acid and acrylic acid, copolymers of acrylamides and (meth)acrylic acid, polysaccharides, such as starch or guar derivatives, gelatin and materials with the INCI designations polyquaternium 2, polyquaternium 17, polyquaternium 18 and polyquaternium 27. The water-soluble material is particularly preferably a polyvinyl alcohol.

In one embodiment of the invention, the water-soluble material comprises mixtures of different substances. Such mixtures make it possible for the mechanical properties of the container to be adjusted and can influence the degree of water solubility.

The water-soluble material preferably contains at least one polyvinyl alcohol and/or at least one polyvinyl alcohol copolymer. “Polyvinyl alcohol” (abbreviated as PVAL or PVA, or occasionally also as PVOH) is the designation for polymers of the general structure

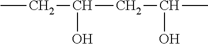

which also contain structural units in small proportions (about 2%) of the type

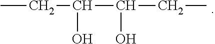

Commercially available polyvinyl alcohols, which are offered as a white-yellowish powder or granulate with degrees of polymerization in the range of from approximately 100 to 2,500 (molar masses of from approximately 4,000 to 100,000 g/mol), have degrees of hydrolysis of from 98 to 99 mol. % or 87 to 89 mol. %, so they still contain residual acetyl groups. The manufacturers characterize the polyvinyl alcohols by stating the degree of polymerization of the starting polymer, the degree of hydrolysis, the saponification number and the solution viscosity.

Depending on the degree of hydrolysis, polyvinyl alcohols are soluble in water and a few strongly polar organic solvents (formamide, dimethylformamide, dimethyl sulfoxide); they are not attacked by (chlorinated) hydrocarbons, esters, fats and oils. Polyvinyl alcohols are classified as toxicologically recognized as safe and are at least partly biodegradable. The water solubility can be reduced by post-treatment using aldehydes (acetalization), by complexing using Ni or Cu salts or by treatment using dichromates, boric acid or borax. The polyvinyl alcohol coatings are largely impervious to gases such as oxygen, nitrogen, helium, hydrogen and carbon dioxide, but allow water vapor to pass through.

In the context of the present invention, it is preferable for the water-soluble material at least partly to comprise a polyvinyl alcohol of which the degree of hydrolysis is from 70 to 100 mol. %, preferably from 80 to 90 mol. %, particularly preferably from 81 to 89 mol. %, and in particular from 82 to 88 mol. %. In a preferred embodiment, the water-soluble material consists of at least 20 wt. %, particularly preferably at least 40 wt. %, very particularly preferably at least 60 wt. %, and in particular at least 80 wt. % of a polyvinyl alcohol, of which the degree of hydrolysis is from 70 to 100 mol. %, preferably 80 to 90 mol. %, particularly preferably 81 to 89 mol. %, and in particular 82 to 88 mol. %.

The polyvinyl alcohols described above are widely available commercially, for example under the trademark Mowiol® (Clariant). Polyvinyl alcohols which are particularly suitable in the context of the present invention are, for example, Mowiole 3-83, Mowiol® 4-88, Mowiol® 5-88, Mowiol® 8-88 and L648, L734, Mowiflex LPTC 221 ex KSE and compounds from Texas Polymers such as Vinex 2034.

Preferred polyvinyl alcohol copolymers include, in addition to vinyl alcohol, dicarboxylic acids as further monomers. Suitable dicarboxylic acids are itaconic acid, malonic acid, succinic acid and mixtures thereof, with itaconic acid being preferred.

Polyvinyl alcohol copolymers which include, in addition to vinyl alcohol, an ethylenically unsaturated carboxylic acid, or the salt or ester thereof, are also preferred. Polyvinyl alcohol copolymers of this kind particularly preferably contain, in addition to vinyl alcohol, acrylic acid, methacrylic acid, acrylic acid ester, methacrylic acid ester or mixtures thereof.

The water solubility of polyvinyl alcohol polymer can be altered by post-treatment with aldehydes (acetalization) or ketones (ketalization). Particularly preferred and, due to their decidedly good solubility in cold water, particularly advantageous polyvinyl alcohols have been produced which can be acetalized or ketalized with the aldehyde or keto groups of saccharides or polysaccharides or mixtures thereof.

Furthermore, the water solubility can be altered and thus set at desired values in a targeted manner by complexing using Ni or Cu salts or by treatment using dichromates, boric acid, or borax. These are preferably not included. PVAL films are largely impermeable to gases such as oxygen, nitrogen, helium, hydrogen and carbon dioxide, but allow water vapor to pass through.

In addition to polyvinyl alcohol, polymers selected from the group comprising acrylic acid-containing polymers, polyacrylamides, oxazoline polymers, polystyrene sulfonates, polyurethanes, polyesters, polyethers, polylactic acid, and/or mixtures of the above polymers may additionally be added to the film material suitable as the water-soluble material.

Suitable water-soluble films for use as the water-soluble material of the water-soluble portion according to the invention are films which are sold by MonoSol LLC under the designation M8630 or M8720. Other suitable films include films named Solublon® PT, Solublon® KA, Solublon® KC or Solublon® KL from Aicello Chemical Europe GmbH or the films VF-HP from Kuraray.

Preferred water-soluble materials are characterized in that they comprise hydroxypropylmethyl cellulose (HPMC) which has a degree of substitution (average number of methoxy groups per anhydroglucose unit of the cellulose) of from 1.0 to 2.0, preferably from 1.4 to 1.9, and has a molar substitution (average number of hydroxypropoxyl groups per anhydroglucose unit of the cellulose) of from 0.1 to 0.3, preferably from 0.15 to 0.25.

Polyvinylpyrrolidones, abbreviated as PVP, are produced by radical polymerization of 1-vinylpyrrolidone. Commercially available PVPs have molar masses in the range of from approx. 2,500 to 750,000 g/mol and are offered as white, hygroscopic powders or as aqueous solutions.

Polyethylene oxides, PEOX for short, are polyalkylene glycols of the general formula

H—[O—CH₂—CH₂]ₙ—OH

which are produced industrially by base-catalyzed polyaddition of ethylene oxide (oxirane) in mostly small water-containing systems which contain small amounts of water and have ethylene glycol as the primer. They usually have molar masses in the range of from approx. 200 to 5,000,000 g/mol, corresponding to degrees of polymerization n of from approx. 5 to >100,000. Polyethylene oxides have an extremely low concentration of reactive hydroxy end groups and only demonstrate weak glycol properties.

Gelatin is a polypeptide (molecular weight: approx. 15,000 to >250,000 g/mol) which is primarily obtained by hydrolysis of the collagen contained in the skin and bones of animals under acidic or alkaline conditions. The amino acid composition of the gelatin largely corresponds to that of the collagen from which it was obtained and varies depending on its provenance. The use of gelatin as a water-soluble coating material is extremely widespread, in particular in pharmaceutics in the form of hard or soft gelatin capsules. Gelatin is used only to a minor extent in the form of films because of its high price in comparison with the above-mentioned polymers.

In the context of the present invention, preference is given to water-soluble materials which comprise a polymer from the group starch and starch derivatives, cellulose and cellulose derivatives, in particular methyl cellulose and mixtures thereof.

Starch is a homoglycan, with the glucose units being linked α-glycosidically. Starch is made up of two components of different molecular weights (MW): of approx. 20 to 30% straight-chain amylose (MW approx. 50,000 to 150,000 g/mol) and 70 to 80% branched-chain amylopectin (MW approx. 300,000 to 2,000,000 g/mol). It also contains small amounts of lipids, phosphoric acid and cations. While the amylose forms long, helical, intertwined chains having approximately 300 to 1,200 glucose molecules due to the binding in the 1,4-position, the chain in amylopectin branches out after an average of 25 glucose building blocks by 1,6 binding to form a branch-like structure with about 1,500 to 12,000 molecules of glucose. In addition to pure starch, starch derivatives which can be obtained from polymer-analogous reactions from starch are also suitable for producing water-soluble containers in the context of the present invention. Chemically modified starches of this type in this case comprise products of esterification or etherification in which hydroxy hydrogen atoms have been substituted. However, starches in which the hydroxy groups have been replaced by radicals which are not bound by an acid atom may also be used as starch derivatives. The group of starch derivatives includes, for example, alkali starches, carboxymethyl starch (CMS), starch esters and ethers and amino starches.

Pure cellulose has the formal gross composition (C₆H₁₀O₅) and is formally considered to be a β-1,4-polyacetal of cellobiose, which itself is composed of two glucose molecules. In this case, suitable celluloses are composed of from approximately 500 to 5,000 glucose units and therefore have an average molecular mass of from 50,000 to 500,000 g/mol. Cellulose derivatives which can be obtained from cellulose by polymer-like reactions may also be used as cellulose-based disintegration agents in the context of the present invention. Chemically modified celluloses of this type in this case comprise products of esterification or etherification in which hydroxy hydrogen atoms have been substituted. However, celluloses in which the hydroxy groups have been replaced by radicals which are not bound by an acid atom may also be used as cellulose derivatives. The group of cellulose derivatives includes, for example, alkali celluloses, carboxymethyl cellulose (CMC), cellulose esters and ethers as well as amino celluloses.

The water-soluble material can have further additives. These are, for example, plasticizers, such as dipropylene glycol, ethylene glycol or diethylene glycol, water or disintegrating agents.

Polyvinyl alcohol is particularly preferably used as the water-soluble material. This is easy to process and inexpensive to maintain. In addition, it is particularly soluble in water and thus makes it possible for the produced container to be used in a variety of ways.

It is preferred according to the invention if at least one bittering agent is contained in the water-soluble material to increase product safety.

Preferred bittering agents have a bitter value of at least 1,000, preferably at least 10,000, particularly preferably at least 200,000. In order to determine the bitter value, the European Pharmacopoeia (5th Edition, Grundwerk, Stuttgart 2005, Volume 1, General Part, Monograph Groups, 2.8.15 bitter value p. 278) uses the standardized procedures described. An aqueous solution of quinine hydrochloride, of which the bitter value is fixed at 200,000, is used as a comparison. This means that 1 gram of quinine hydrochloride makes 200 liters of water bitter. The inter-individual taste differences in the organoleptic bitterness test are compensated for by a correction factor in this method.

Very particularly preferred bittering agents are selected from denatonium benzoate, glycosides, isoprenoids, alkaloids, amino acids, and mixtures thereof, particularly preferably denatonium benzoate.

Glycosides are organic compounds of the general structure R—O—Z, in which an alcohol (R—OH) is linked to a sugar part (Z) by means of a glycosidic bond.

Suitable glycosides are, for example, flavonoids such as quercetin or naringin or iridoidglycoside such as aucubin and in particular secoiridoid, such as amarogentin, dihydrofoliamentin, gentiopicroside, gentiopikrin, swertiamarin, sweroside, gentioflavoside, centauroside, methiafolin, harpagoside and centapikrin, sailicin or condurangin.

Isoprenoids are compounds that are formally derived from isoprene. Examples are in particular terpenes and terpenoids.

Suitable isoprenoids comprise, for example, sequiterpene lactones such as absinthin, artabsin, cnicin, lactucin, lactucopikrin or salonitenolide, monoterpene ketones (thujones) such as α-thujone or β-thujone, tetranortriterpenes (limonoids) such as deoxylimones, desoxylimonic acid, limonin, ichangin, iso-obacunonic acid, obacunone, obacunonic acid, nomilin or nomilic acid, terpenes such as marrubin, premarrubin, carnosol, carnosolic acid or quassin.

Alkaloids refer to naturally occurring, chemically heterogeneous, mostly alkaline, nitrogen-containing organic compounds of secondary metabolism that act on the animal or human organism.

Suitable alkaloids are, for example, quinine hydrochloride, quinine hydrogen sulfate, quinine dihydrochloride, quinine sulfate, columbine and caffeine.

Suitable amino acids comprise, for example, threonine, methionine, phenylalanine, tryptophan, arginine, histidine, valine and aspartic acid.

Particularly preferred bitterns are quinine sulfate (bitter value=10,000), naringin (bitter value=10,000), sucrose octaacetate (bitter value=100,000), quinine hydrochloride, denatonium benzoate (bitter value>100,000,000) and mixtures thereof, very particularly preferably denatonium benzoate (for example available as Bitrex®).

Based on the total weight thereof, the water-soluble material preferably contains bittering agents (particularly preferably denatonium benzoate) in a total amount of at most 1 part by weight bitter matter to 250 parts by weight viscoelastic, solid composition (1:250), particularly preferably at most 1:500, very particularly preferably of at most 1:1,000, based on the total weight of said shell material.

It is preferred to use the composition for washing textiles, in particular for removing soiling that is based on components and residues of deodorants, rust, berries, tea and red wine.

As already mentioned, the stain can be pretreated using the composition according to the invention before the actual washing process and/or initially a surfactant-containing liquor is provided as a washing solution which contains the multi-component detergent according to the invention and is then brought into contact with the textiles to be cleaned.

Methods for cleaning textiles are generally characterized by the fact that, in a plurality of method steps, various cleaning-active substances are applied to the material to be cleaned and washed off after the exposure time, or in that the material to be cleaned is otherwise treated with a detergent or a solution of the composition.

In the washing process described, temperatures of 60° C. or less, for example 40° C. or less, are used in different embodiments of the invention. This temperature information relates to the temperatures used in the washing steps.

It is also preferred to carry out the method according to the invention in a washing machine. It has been found to be particularly effective and therefore preferred if the said composition is dosed into the drum of a washing machine.

All of the embodiments described herein in connection with the detergent of the invention, in particular with regard to the specification of the ingredients, are equally applicable to the methods and uses described and vice versa.

The following aspects illustrate the invention by way of example without restricting it:
1. Surfactant composition, in particular laundry detergents, which comprise at least one catechol compound of the formula (I)

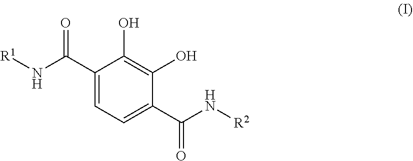

wherein
R¹ and R² represent, independently of one another, a hydrocarbon radical having 1 to 20 carbon atoms that is optionally substituted by at least one radical selected from hydroxy, (C₁-C₄)-alkoxy, (C₁-C₄)-alkoxy(CH₂CH₂O)ₙ—, —NR′R″ or —NTR′R″R′″X⁻, wherein n=1 to 10, R′, R″ and R′″ represent, independently of one another, H or a linear or branched aliphatic hydrocarbon radical having 1 to 3, preferably 1 to 2, carbon atoms and X⁻ represents an anion,
at least one surfactant, preferably in a total amount of from 2 to 70 wt. % relative to the total weight, in particular 10 to 65 wt. %, particularly preferably 15 to 60 wt. %.;
at least one mannanase enzyme;
optionally other active ingredients; and
water based on the total weight of the surfactant composition from 1 to 50 wt. %, preferably 2 to 30 wt. %, particularly preferably 3 to 25 wt. %, very particularly preferably 5 to 25 wt. %, in particular 5 to 35 wt. %, preferably 5 to 30 wt. %, particularly preferably 5 to 25 wt. %, very particularly preferably 8 to 15 wt. %.
2. Surfactant composition according to aspect 1, characterized in that in formula (I) the radicals R¹ and R² represent, independently of one another, an alkyl group, an alkoxyalkyl group, a hydroxyalkyl group, a hydroxyalkyloxyalkyl group, (N-hydroxyethyl)-aminoethyl, (N-methoxyethyl)-aminoethyl or (N-ethoxyethyl)-aminoethyl, or an aromatic group.
3. Surfactant composition according to one of aspects 1 or 2, characterized in that in formula (I), the radicals R¹ and R² are identical.
4. Surfactant composition according to one of aspects 1 to 3, characterized in that, based on its total weight, the at least one catechol compound of the formula (I) is contained in a total amount of 0.2 to 90 wt. %, preferably 1 to 85 wt. %, in particular 2.5 to 75 wt. %.
5. Surfactant composition according to one of aspects 1 to 4, characterized in that it has a pH of 3 to 11.5, preferably 5 to 9, more preferably 7, at 20° C.
6. Surfactant composition according to one of aspects 1 to 5, characterized in that it contains at least one anionic surfactant and/or at least one non-ionic surfactant.
7. Surfactant composition according to one of aspects 1 to 6, characterized in that it contains, based on its total weight, mannanase enzyme in a total amount of 0.01 to 2.5 wt. %, in particular 0.02 to 1.0 wt. %.
8. Surfactant composition according to any of aspects 1 to 7, characterized in that the mannanase enzyme contains at least one mannanase polypeptide from gram-positive alkalophilic strains of Bacillus, in particular selected from at least one representative of the group of Bacillus subtilis, Bacillus lentus, Bacillus clausii, Bacillus agaradhaerens, Bacillus brevis, Bacillus stearothermophilus, Bacillus alkalophilus, Bacillus amyloliquefaciens, Bacillus coagulans, Bacillus circulans, Bacillus lautus, Bacillus thuringiensis, Bacillus cheniformis, and Bacillus sp., particularly preferably selected from at least one representative of the group of Bacillus sp. 1633, Bacillus sp. AAI12, Bacillus clausii, Bacillus agaradhaerens and Bacillus licheniformis.
9. Surfactant composition according to any of aspects 1 to 8, characterized in that the mannanase enzyme is selected from at least one representative of the group formed by polypeptides which comprise an amino acid sequence of which the sequence is at least 90% (in order of increasing preference at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7% or 99.8%) identical to the polypeptide of the positions 31 to 490 according to SEQ ID no. 1.
10. Surfactant composition according to one of aspects 1 to 8, characterized in that it contains
at least one mannanase from Paenibacillus sp. or
one of the variants thereof, comprising an amino acid sequence having at least two changes selected from
   (i) at least one substitution in at least one of the positions selected from 1, 2,
   3, 4, 6, 10, 19, 28, 30, 38, 59, 60, 61, 62, 63, 66, 67, 68, 70, 71, 74, 75, 78, 80, 82, 93, 97, 103, 111, 124, 129, 131, 135, 136, 139, 143, 150, 167, 168, 184, 213, 214, 217, 225, 228, 235, 242, 244, 258, 259, 261, 283 and 284, and
   (ii) an insertion at position 298, wherein said amino acid positions corresponds to the positions according to SEQ ID no. 2 of this application. SEQ ID no.: 2.
11. Surfactant composition according to any of aspects 1 to 8, characterized in that it is liquid.
12. Portion of a multi-component detergent comprising at least two components and a multi-chamber container, wherein said components are contained separately from one another in a chamber of the multi-chamber container, characterized in that the sum of the components results in a surfactant composition according to any of aspects 1 to 11.
13. Portion according to aspect 12, characterized in that the multi-chamber container is in the form of a multi-chamber pouch, in particular having a water-soluble film, particularly preferably based on polyvinyl alcohol.
14. Portion according to any of aspects 11 or 12, characterized in that it contains at least one bitter substance, and the bitter substance is preferably contained in the surfactant composition and/or in the material of the multi-chamber container, in particular in the material of the water-soluble film of the multi-chamber container.
15. Use of the surfactant composition according to any of aspects 1 to 11 or the portion according to any of aspects 12 to 14 for removing bleachable soiling.
16. Method for washing textiles, comprising the steps of:
   Adding a surfactant composition according to any of aspects 1 to 11 or a portion according to any of aspects 12 to 14 to a fabric or textile; and performing a washing procedure, preferably in a washing machine.

### Examples

1.0 Provision of the Test Compositions
The following liquid detergents were produced:

| | V1 | V2 | E1 |
|---|---|---|---|
| | (comparison) | (comparison) | (invention) |
| | wt. % (active | wt. % (active | wt. % (active |
| Ingredient | substance) | substance) | substance) |
| Propylene glycol | 8.2 | 8.2 | 8.2 |
| Glycerol | 10.5 | 10.5 | 10.5 |
| Optical brightener | 0.6 | 0.6 | 0.6 |
| Linear alkyl benzene sulfonate | 22.0 | 22.0 | 22.0 |
| C₁₃₋₁₅ oxo alcohol with 8 EO units | 24.0 | 24.0 | 24.0 |
| 2-aminoethanol | 6.0 | 6.0 | 6.0 |
| C₁₂₋₁₈ soap sodium salt | 7.5 | 7.5 | 7.5 |
| Ethoxylated polyethylenimine polymer | 6.0 | 6.0 | 6.0 |
| (PEI 600 with 20 EO) | | | |
| Diethylenetriaminepenta(methylenephosphonic | 0.7 | 0.7 | 0.7 |
| acid)-heptasodium salt (DTPMPA-7 sodium | | | |
| salt) | | | |
| Ethanol | 3.0 | 3.0 | 3.0 |
| N,N′-Dipropyl-2,3-dihydroxyterephthaldiamide ¹ | 0.5 | 0.5 | 0.5 |
| Soil release polymer (tephthalic acid polyester) | 1.4 | 1.4 | 1.4 |
| Perfume | 1.7 | 1.7 | 1.7 |
| Catechol derivative ¹ | 1.5 | — | 1.5 |
| Mannanase according to SEQ ID no. 1 ² | — | 0.04 | 0.04 |
| Water | to make up | to make up | to make up |
| | to 100 | to 100 | to 100 |

¹ Catechol compound according to the invention of the formula (I) where R¹ and R² are n-propyl, prepared using n-propylamine analogously to the procedure of Example 2 of WO 2016/074936 A1.
² Sequence identity to the polypeptide of positions 31 to 490 according to SEQ ID no. 1

2.0 Washing Test
Washing tests were carried out at 40° C. with the formulations V1, V2 and E1 given in the table above (dosage 25 g to 171 of water at 16° dH) with standardized “Mousse au chocolat” soiling on cotton.

Before washing and after drying the washed cotton rags, their brightness was determined with the aid of color distance measurement according to the L*a*b* values, and the Y values were determined as a measure of the brightness. The higher the Y value, the closer the value of the laundry after washing to the initial value without dirt.

The following table shows the determined Y values.

Y values after the washing test:

| Soiling | V1 | V2 | E1 |
|---|---|---|---|
| Mousse au Chocolat | 47.9 | 48.8 | 50.2 |

What is claimed is:

1. A surfactant composition comprising:
at least one catechol compound of the formula (I) 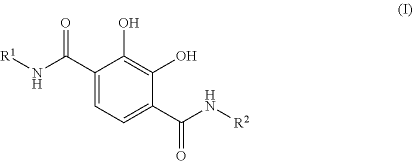
wherein
R¹ and R² represent, independently of one another, a hydrocarbon radical having 1 to 20 carbon atoms that is optionally substituted by at least one radical selected from hydroxy, (C₁-C₄)-alkoxy, (C₁-C₄)-alkoxy(CH₂CH₂O)ₙ—, —NR′R″ or —N⁺R′R″R′″ X⁻, wherein n=1 to 10, R′, R″ and R′″ represent, independently of one another, H or a linear or branched aliphatic hydrocarbon radical having 1 to 3 carbon atoms and X⁻ represents an anion,
at least one surfactant, in a total amount from 2 to 70 wt. % relative to the total weight;
at least one mannanase enzyme;
optionally other active ingredients; and
water based on the total weight of the surfactant composition from 1 to 50 wt. %.

2. The surfactant composition according to claim 1, wherein in formula (I) the radicals R¹ and R² represent, independently of one another, an alkyl group, an alkoxyalkyl group, a hydroxyalkyl group, a hydroxyalkyloxyalkyl group, (N-hydroxyethyl)-aminoethyl, (N-methoxyethyl)-aminoethyl or (N-ethoxyethyl)-aminoethyl, or an aromatic group.

3. The surfactant composition according to claim 1, wherein in formula (I), the radicals R¹ and R² are identical.

4. The surfactant composition according to claim 1, wherein, based on its total weight, the at least one catechol compound of the formula (I) is contained in a total amount of 0.2 to 90 wt. %.

5. The surfactant composition according to claim 1, wherein it contains at least one anionic surfactant and/or at least one non-ionic surfactant.

6. The surfactant composition according to claim 1, wherein, based on its total weight, mannanase enzyme is contained in a total amount of 0.01 to 2.5 wt. %.

7. The surfactant composition according to claim 1, wherein the mannanase enzyme contains at least one mannanase polypeptide from gram-positive alkalophilic strains of Bacillus.

8. The surfactant composition according to claim 1, wherein the mannanase enzyme is selected from at least one representative of the group formed by polypeptides which comprise an amino acid sequence of which the sequence is at least 90% identical to the polypeptide of the positions 31 to 490 according to SEQ ID no. 1.

9. The surfactant composition according to claim 1, wherein it contains:
at least one mannanase from Paenibacillus sp. Or
one of the variants thereof, comprising an amino acid sequence having at least two changes selected from
(i) at least one substitution at at least one of the positions selected from 1, 2, 3, 4, 6, 10, 19, 28, 30, 38, 59, 60, 61, 62, 63, 66, 67, 68, 70, 71, 74, 75, 78, 80, 82, 93, 97, 103, 111, 124, 129, 131, 135, 136, 139, 143, 150, 167, 168, 184, 213, 214, 217, 225, 228, 235, 242, 244, 258, 259, 261, 283 and 284, and
(ii) an insertion at position 298, wherein said amino acid positions corresponds to the positions according to SEQ ID no. 2 of this application. SEQ ID no. 2.

10. A method for washing textiles, comprising the steps of adding a surfactant composition according to claim 1 and carrying out a washing procedure.

11. The surfactant composition, according to claim 1, is a textile detergent.

12. The surfactant composition, according to claim 1, wherein in formula (I), R′, R″ and R′″ represent, independently of one another, H or a linear or branched aliphatic hydrocarbon radical having 1 to 2 carbon atoms.

13. The at least one surfactant of the surfactant composition, according to claim 1, is in a total amount from 15 to 60 wt. % relative to the total weight.

14. The surfactant composition according to claim 4, wherein, based on its total weight, the at least one catechol compound of the formula (I) is contained in a total amount of 1 to 85 wt. %.

15. The surfactant composition according to claim 4, wherein, based on its total weight, the at least one catechol compound of the formula (I) is contained in a total amount of 2.5 to 75 wt. %.

16. The surfactant composition according to claim 6, wherein, based on its total weight, mannanase enzyme is contained in a total amount of 0.02 to 1.0 wt. %.

17. The surfactant composition according to claim 7, wherein the mannanase enzyme contains at least one mannanase polypeptide selected from at least one representative of the group of Bacillus subtilis, Bacillus lentus, Bacillus clausii, Bacillus agaradhaerens, Bacillus brevis, Bacillus stearothermophilus, Bacillus alkalophilus, Bacillus amyloliquefaciens, Bacillus coagulans, Bacillus circulans, Bacillus lautus, Bacillus thuringiensis, Bacillus cheniformis, and Bacillus sp.

18. The surfactant composition according to claim 7, wherein the mannanase enzyme contains at least one mannanase polypeptide selected from at least one representative of the group of Bacillus sp. 1633, Bacillus sp. AAI12, Bacillus clausii, Bacillus agaradhaerens and Bacillus licheniformis.

19. The method for washing textiles and the washing procedure, according to claim 10, is carried out in a washing machine.


SEQUENCE LISTING (2 sequences)

SEQ ID NO: 1 - protein, 490 aa, Bacillus sp.
LNNGFKKIFSITLSLLLASSILFVSGTSTANANSGFYVSGTTLYDANGNPFVMRGINHGHAWYKDQATTAIEGIANTGANTVRIVLSDGGQWTKDDIHTVRNLISLAEDNHLVAVPEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSPTTLYDFEGSMQGWTGSSLSGGPWAVTEWSSKGSHSLKADIQLSSNSQHYLHVIQNTSLQQNSRIQATVKHANWGSVGNGMTARLYVKTGHGYTWYSGSFVPINGSSGTTLSLDLSNVQNLSQVREIGVQFQSASDSSGQTSIYIDNVIVE

SEQ ID NO: 2 - protein, 297 aa, Paenibacillus sp.
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF